SEQUENCE LISTING (22 sequences)

SEQ ID NO: 1 - protein, 31 aa, Artificial sequence
  Synthetic polypeptide
CQYIKANSKFIGITEFGFPEHLLVDFLQSLS

SEQ ID NO: 2 - protein, 28 aa, Artificial sequence
  Synthetic polypeptide
XKXVAAWTLKAXFGFPEHLLVDFLQSLS

SEQ ID NO: 3 - DNA, 24 bp, Artificial sequence
  Synthetic oligonucleotide
tcgtcgttttgtcgttttgtcgtt

SEQ ID NO: 4 - DNA, 34 bp, Artificial sequence
  Synthetic oligonucleotide
tcnnnnnnnnnnnnnnnnnnnnnnnnntnncgnn

SEQ ID NO: 5 - DNA, 12 bp, Artificial sequence
  Synthetic oligonucleotide
cgacgttcgtcg

SEQ ID NO: 6 - DNA, 13 bp, Artificial sequence
  Synthetic oligonucleotide
cggcgccgtgccg

SEQ ID NO: 7 - DNA, 12 bp, Artificial sequence
  Synthetic oligonucleotide
ccccccgggggg

SEQ ID NO: 8 - DNA, 12 bp, Artificial sequence
  Synthetic oligonucleotide
ggggggcccccc

SEQ ID NO: 9 - DNA, 10 bp, Artificial sequence
  Synthetic oligonucleotide
cccccggggg

SEQ ID NO: 10 - DNA, 10 bp, Artificial sequence
  Synthetic oligonucleotide
gggggccccc

SEQ ID NO: 11 - DNA, 21 bp, Artificial sequence
  Synthetic oligonucleotide
tcgcgtcgttcggcgcgcgcc

SEQ ID NO: 12 - DNA, 23 bp, Artificial sequence
  Synthetic oligonucleotide
tcgtcgacgttcggcgcgcgccg

SEQ ID NO: 13 - DNA, 21 bp, Artificial sequence
  Synthetic oligonucleotide
tcggacgttcggcgcgcgccg

SEQ ID NO: 14 - DNA, 19 bp, Artificial sequence
  Synthetic oligonucleotide
tcggacgttcggcgcgccg

SEQ ID NO: 15 - DNA, 20 bp, Artificial sequence
  Synthetic oligonucleotide
tcgcgtcgttcggcgcgccg

SEQ ID NO: 16 - DNA, 20 bp, Artificial sequence
  Synthetic oligonucleotide
tcgacgttcggcgcgcgccg

SEQ ID NO: 17 - DNA, 18 bp, Artificial sequence
  Synthetic oligonucleotide
tcgacgttcggcgcgccg

SEQ ID NO: 18 - DNA, 18 bp, Artificial sequence
  Synthetic oligonucleotide
tcgcgtcgttcggcgccg

SEQ ID NO: 19 - DNA, 22 bp, Artificial sequence
  Synthetic oligonucleotide
tcgcgacgttcggcgcgcgccg

SEQ ID NO: 20 - protein, 21 aa, Clostridium tetani
FNNFTVSFWLRVPKVSASHLE

SEQ ID NO: 21 - protein, 21 aa, Homo sapiens
CSKGTSHEAGIVCRITKPALL

SEQ ID NO: 22 - protein, 20 aa, Homo sapiens
SKGTSHEAGIVCRITKPALL

(19) United States
(12) Patent Application Publication
(10) Pub. No.: US 2009/0142362 A1
(43) Pub. Date: Jun. 4, 2009

(54) Peptide-based vaccine compositions to endogenous cholesteryl ester transfer protein (CETP)

(75) Inventors: Arthur M. Krieg, Wellesley, MA (US); Lawrence J. Thomas, South Easton, MA (US); Charles W. Rittershaus, Malden, MA (US)

Correspondence Address: WOLF GREENFIELD & SACKS, P.C., 600 ATLANTIC AVENUE, BOSTON, MA, 02210-2206, US

(73) Assignees: Avant Immunotherapeutics, Inc., Needham, MA (US); Coley Pharmaceutical Group, Inc., Wellesley, MA (US)

(21) Appl. No.: 11/983,039

(22) Filed: Nov. 6, 2007

Related U.S. Application Data
(60) Provisional application No. 60/859,005, filed on Nov. 6, 2006.

(51) Int. Cl. A61K 39/00; A61K 39/08; A61K 39/05; A61K 39/10; A61K 39/13; A61K 39/165; A61K 39/20

(52) U.S. Cl. 424/185.1; 424/184.1; 424/239.1; 424/238.1; 424/254.1; 424/217.1; 424/212.1; 424/219.1

(57) ABSTRACT

Improved vaccine compositions and methods of use thereof are described that elicit production of antibodies in an individual to the individual's own endogenous cholesteryl ester transfer protein (CETP).

### RELATED APPLICATION

This application claims priority under 35 USC § 119 to U.S. Provisional Application No. 60/859,005, filed Nov. 6, 2006, the entire contents of which is hereby incorporated by reference.

### BACKGROUND OF INVENTION

Cholesterol circulates in the blood associated with a variety of lipoprotein molecules that are classically defined with respect to their relative densities, such as, high density lipoprotein-associated cholesterol (“HDL-C”, so called “good cholesterol”), low density lipoprotein-associated cholesterol (“LDL-C”, a so-called “bad cholesterol”), and very low density lipoprotein-associated cholesterol (“VLDL-C”, another so-called “bad cholesterol”). Susceptibility to and decreased risk of cardiovascular disease, such as atherosclerosis, is generally correlated with a profile of one or more levels of such lipoprotein-associated cholesterol molecules. For example, the type of changes in the profile of such lipoprotein-associated cholesterol molecules that are generally correlated with a healthier cardiovascular condition and/or decreased risk of atherosclerosis include an increase in the absolute level of HDL-C; an increase in the ratio of HDL-C to LDL-C, VLDL-C, or total cholesterol; a decrease in the absolute level of serum LDL-C; and combinations thereof.

In humans, cholesteryl ester transfer protein (CETP) is a hydrophobic plasma glycoprotein that has 476 amino acids and a molecular weight of approximately 66,000 to 74,000 daltons (see, e.g., Hesler et al., J. Biol. Chem., 262: 2275-2282 (1987)). CETP mediates the transfer of plasma cholesteryl esters from high density lipoprotein (HDL) to triglyceride (TG)-rich lipoproteins such as low density lipoprotein (LDL) and very low density lipoprotein (VLDL), and also the reciprocal exchange of TG from VLDL to HDL (Hesler et al., 1987). The region of CETP defined by the carboxyl terminal 26 amino acids has been shown to be especially important for neutral lipid binding involved in neutral lipid transfer (see, e.g., Hesler et al, J. Biol. Chem., 263: 5020-5023 (1988)). Thus, CETP appears to play to a major role in modulating the levels of cholesteryl esters and TG that are associated with the various classes of lipoproteins. A high CETP activity has been correlated with increased levels of LDL-C and VLD-C, which in turn have been correlated with increased risk of cardiovascular disease (see, e.g., Tato et al., Arterioscler. Thromb. Vascular Biol., 15:112-120 (1995)). Thus, inhibiting endogenous CETP activity is an attractive therapeutic approach for modulating the relative levels of lipoprotein-associated cholesterol molecules for the treatment or prevention of atherosclerosis.

A promising new approach has emerged for the treatment and prevention of atherosclerosis that is based on directing an individual's immune system to produce autoantibodies that inhibit the activity of the individual's endogenous CETP. Thus, both immunogenic peptide-based (see, e.g., U.S. Pat. No. 6,410,022 and U.S. Pat. No. 6,555,113) and immunogenic plasmid-based (see, e.g., U.S. Pat. No. 6,284,533 and U.S. Pat. No. 6,846,808) vaccine compositions have been described that inhibit endogenous CETP activity for the treatment or prevention of atherosclerosis. An example of the antigenic hybrid peptides described in U.S. Pat. No. 6,410,022 comprise a universal (or “broad range”) helper T cell epitope peptide (e.g., a peptide of the tetanus toxoid) linked to a B cell epitope-containing peptide from the carboxyl terminal 16 amino acids of human CETP. When administered to a mammalian individual, such peptides are “autoimmunogenic” and elicit the production of antibodies that recognize (bind to) the individual's endogenous (native) CETP, leading to a decrease in CETP activity. Data presented in U.S. Pat. No. 6,410,022 demonstrated that administering such antigenic peptides to test animals led inter alia to a rise in the level of circulating HDL-C, a rise in the ratio of HDL-C to LDL-C or VLDL-C, a lowering of the level of circulating total cholesterol, and a significant reduction in the development of atherosclerotic lesions in arteries of rabbits in a model for atherosclerosis featuring a high-cholesterol diet.

The foregoing developments are very promising for the development of an alternative approach to various statin drugs that have been approved for controlling cholesterol metabolism and addressing cardiovascular disease. Thus, interest and needs remain for advancements in the field of vaccines that elicit a directed autoimmunity against endogenous CETP for treating and preventing cardiovascular disease.

### SUMMARY OF INVENTION

The invention described herein provides improved peptide-based vaccine compositions that, when administered to an individual (human or other mammal), elicit production of antibodies in the individual that recognize (bind to) endogenous cholesteryl ester transfer protein (CETP), which elicited antibodies are produced at significantly and unexpectedly higher levels than were heretofore obtained using previously described CETP vaccine compositions.

In one embodiment, the invention provides a vaccine composition for eliciting antibodies in an individual against the individual's own, endogenous cholesteryl ester transfer protein (CETP) comprising:
(a) an antigenic hybrid polypeptide comprising a B cell epitope portion linked to a universal helper T cell epitope portion, wherein said B cell epitope portion comprises a B cell epitope of said endogenous CETP, and wherein said universal helper T cell epitope portion comprises a broad range helper T cell epitope that binds multiple class II major histocompatibility complex (MHC) alleles expressed on antigen-presenting cells, and is, preferably, a non-naturally occurring amino acid sequence that binds multiple DR alleles expressed on antigen-presenting cells, and
(b) an adjuvant comprising an immunostimulatory oligonucleotide.

In some embodiments the immunostimulatory oligonucleotide is a CpG oligonucleotide having at least one unmethylated CpG dinucleotide.

In a preferred embodiment, the B cell epitope portion of a CETP vaccine composition described herein comprises a B cell epitope of human CETP and has the amino acid sequence:

| | |
|---|---|
| FGFPEHLLVDFLQSLS. | (amino acids 16-31 of |
| | SEQ ID NO: 1) |

In another embodiment, a CETP vaccine composition described herein comprises a universal helper T cell epitope portion that comprises any of a variety of peptides that bind multiple class II major histocompatibility complex (MHC) alleles expressed on antigen-presenting cells, including helper T cell epitope peptides derived from naturally occurring broad range immunogenic peptides such as tetanus toxoid, diphtheria toxoid, pertussis vaccine, Bacile Calmette-Guerin (BCG), polio vaccine, measles vaccine, mumps vaccine, rubella vaccine, purified protein derivative of tuberculin, keyhole limpet hemocyanin, hsp70, and combinations thereof.

More preferably, the universal helper T cell epitope portion of a CETP vaccine composition described herein comprises a broad range helper T cell epitope that is a universal helper T cell epitope peptide from tetanus toxin that has the amino acid sequence:

| | |
|---|---|
| QYIKANSKFIGITE. | (nucleotides 2-15 of SEQ ID NO: 1) |

In another embodiment, the universal helper T cell epitope portion of a CETP vaccine composition described herein comprises a broad range helper T cell epitope peptide that has a non-naturally occurring amino acid sequence and that binds multiple DR alleles expressed on antigen-presenting cells. More preferably, the broad range helper T cell epitope has the amino acid sequence:

| | |
|---|---|
| aKChaVAAWTLKAa, | (amino acids 1-12 of SEQ ID NO: 2) |

wherein “a” is D-alanine and “Cha” is cyclohexylalanine.

In still another embodiment, the terminal amino acid of the antigenic hybrid polypeptide of a vaccine composition described herein has an alpha carboxylamide group.

In some embodiments the immunostimulatory oligonucleotide is conjugated to the antigenic hybrid polypeptide. In other embodiments the immunostimulatory oligonucleotide and the antigenic hybrid polypeptide are linked. The linkage may be direct or indirect. An indirect linkage includes, for instance, formulation in a single carrier causing close association of the two components.

According to some embodiments, the oligonucleotide is 3-100 nucleotides in length; for example, the oligonucleotide may be 3-6 nucleotides in length, 3-80 nucleotides in length, or 7-50 nucleotides in length. In some circumstances, the oligonucleotide is T-rich, such that at least 80% of the nucleotides are T.

According to the invention, some embodiments include one to four unmethylated CG dinucleotides. In some cases, at least one but up to all CG dinucleotides are unmethylated. According to some embodiments, the oligonucleotide may additionally comprise a non-nucleotidic modification. The non-nucleotidic modifications include but are not limited to: C₆-C₄₈-polyethyleneglycol, C₃-C₂₀-alkane-diol, C₃-C₁₈-alkylamino linker, C₃-C₁₈-alkylthiol linker, cholesterol, bile acid, saturated or unsaturated fatty acid, folate, a hexadecyl-glycerol or dihexadecyl-glycerol group, an octadecyl-glycerol or dioctadecyl-glycerol group, a vitamin E group. In other embodiments, the oligonucleotide of the invention further comprises a non-nucleotidic brancher moiety or a nucleotidic brancher moiety. In some embodiments, the oligonucleotide includes a brancher moiety, wherein the oligonucleotides has at least two 5′-ends.

According to the invention, some embodiments include oligonucleotides wherein at least two nucleotides have a stabilized linkage, including: phosphorothioate, phosphorodithioate, methylphosphonate, methylphosphonothioate boranophosphonate, phosphoramidate, or a dephospho linkage, either as enantiomeric mixture or as enantiomeric pure S- or R-configuration.

Yet in some embodiments, one or more of the CG dinucleotides have a phosphodiester linkage or a phosphorothioate linkage. In some embodiments, all other nucleotides have a phosphorothioate linkage.

In some embodiments, the oligonucleotides may be CpG oligonucleotides such as an A class oligonucleotide, a B class oligonucleotide, a C class oligonucleotide, a P class oligonucleotide or a T class oligonucleotide. For the B class oligonucleotide of the invention, some embodiments include the sequence 5′ TCN₁TX₁X₂CGX₃X₄ 3′, wherein X₁ is G or A; X₂ is T, G, or A; X₃ is T or C and X₄ is T or C; and N is any nucleotide, and N₁ and N₂ are nucleic acid sequences of about 0-25 N's each.

In other embodiments the immunostimulatory oligonucleotide is an RNA oligonucleotide. The RNA oligonucleotide may be, for instance, 5′-C/U-U-G/U-U-3′, 5′-R-U-R-G-Y-3′, 5′-G-U-U-G-B-3′, 5′-G-U-G-U-G/U-3′, or 5′-G/C-U-A/C-G-G-C-A-C-3′, wherein C/U is cytosine (C) or uracil (U), G/U is guanine (G) or U, R is purine, Y is pyrimidine, B is U, G, or C, G/C is G or C, and A/C is adenine (A) or C. In some embodiments, 5′-C/U-U-G/U-U-3′ is CUGU, CUUU, UUGU, or UUUU. In other embodiments 5′-R-U-R-G-Y-3′ is GUAGU, GUAGC, GUGGU, GUGGC, AUAGU, AUAGC, AUGGU, or AUGGC. The RNA oligonucleotides may be for instance, GUAGUGU or GUGUUUAC. In other embodiments 5′-G/C-U-A/C-G-G-C-A-C-3′ is GUAGGCAC, GUCGGCAC, CUAGGCAC, or CUCGGCAC.

According to some embodiments of the invention, the oligonucleotide comprises at least one 3′-3′ linkage and or at least one 5′-5′ linkage.

In yet other embodiments the CpG oligonucleotide is 5′TCGTCGTTTTGTCGTTTTGTCGTT3′ (SEQ ID NO.: 3), wherein, optionally, all nucleotides of the CpG oligonucleotide have a phosphorothioate linkage.

A CETP vaccine composition described herein may be administered to an individual to inhibit or reduce circulating CETP activity in the individual, to alter the level of one or more lipoprotein-associated cholesterol molecules in the blood of the individual, and to treat the development of atherosclerosis in the individual.

Preferably, a CETP vaccine composition described herein is administered to an individual parenterally, including, but not limited to, subcutaneously (s.c.), intramuscularly (i.m.), intravenously (i.v.), intradermally (i.d.), intraperitoneally (i.p.), and intra-arterially (i.a). Other routes of administration useful according to the methods of the invention include but are not limited to sublingual, intratracheal, inhalation and mucosal routes such as oral, intranasal, ocular, vaginal, and rectal. More preferably, a CETP vaccine composition is administered to a human individual subcutaneously or intravenously.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a bar graph illustrating the surprisingly high titers of autoantibodies obtained using a CETP vaccine composition according to the invention, in comparison to vaccine compositions known, e.g., from U.S. Pat. No. 6,410,022.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Cholesteryl ester transfer protein (CETP) has been validated as a therapeutic target for raising the ratio of cholesterol associated with high density lipoproteins (“HDL-C”, so called “good cholesterol”) to cholesterol associated with low density lipoproteins (“LDL-C”, so-called “bad cholesterol”) and/or with very low density lipoproteins (“VLDL-C”, another so-called “bad cholesterol”), for raising the level of circulating HDL-C, and for treating or preventing atherosclerosis (see, e.g., Davidson et al., Atherosclerosis, 169(1): 113-117 (2003); U.S. Pat. No. 6,410,022; U.S. Pat. No. 6,555,113).

As with previously described CETP vaccines, the CETP vaccine compositions described herein are designed to be administered to an individual to elicit a directed immune response to the individual's own CETP, i.e., to elicit an autoimmune response that specifically targets the endogenous CETP circulating in the individual. Unlike previously described CETP vaccines, the vaccine compositions described herein elicit significantly and unexpectedly higher levels of antibodies (titers) to CETP. For example, in a rabbit model used to assess CETP vaccines, significantly higher levels of antibodies are produced when the broad range helper T cell epitope TT 830-843 of tetanus toxoid (Valmori et al., J. Immunol., 149: 717-721 (1992)), which was employed in previously described CETP vaccines, is replaced with a pan DR epitope or when the CpG oligonucleotide adjuvant of SEQ ID NO. 3 (Coley Pharmaceuticals, Inc., Wellesley, Mass.) is incorporated into a vaccine composition. In particular, in the rabbit model, vaccine compositions comprising either a pan DR epitope or the CpG oligonucleotide (SEQ ID NO. 3) adjuvant produce levels of antibodies to CETP that are at least 5 to 6-fold higher than those found with vaccine compositions that lack either of these elements (see, Example, below). Moreover, CETP vaccine compositions as described herein that comprise both a pan DR epitope and the CpG oligonucleotide (SEQ ID NO. 3) adjuvant elicit levels of anti-CETP antibodies that are significantly greater than the sum of the effect of each element alone (see, Example, below). In particular, CETP vaccine compositions comprising both elements provide a desirable and unexpected synergistic effect in the production of anti-CETP antibodies. In addition, the elevated levels (titers) of anti-CETP antibodies elicited by the vaccine compositions described herein are produced without evidence of an accompanying generalized breakdown of tolerance to other self-antigens (reactogenicity).

The invention provides vaccine compositions for eliciting antibodies in an individual to endogenous cholesteryl ester protein (CETP) comprising:
(a) an antigenic hybrid polypeptide comprising a B cell epitope portion linked to universal helper T cell epitope portion, wherein said B cell epitope portion comprises a B cell epitope of said endogenous CETP, and wherein said universal helper T cell epitope portion comprises a broad range helper T cell epitope that binds multiple class II major histocompatibility complex (MHC) alleles expressed on antigen-presenting cells, and
(b) an adjuvant comprising an immunostimulatory oligonucleotide.

The immunostimulatory oligonucleotide may be a CpG oligonucleotide having at least one unmethylated CpG dinucleotide.

Preferably the universal helper T cell epitope portion comprises a non-naturally occurring amino acid sequence and binds multiple DR alleles expressed on antigen-presenting cells, known as a pan-DR epitope (PADRE) sequence.

In order to more fully understand the invention, the following abbreviations, terms, and phrases are defined for use in the description of the invention:

Abbreviations: L-Amino acid residues described herein may be abbreviated by their conventional three-letter or one-letter abbreviations (see, e.g., Lehninger, A. L., Biochemistry, second edition (Worth Publishers, Inc., New York, 1975), p. 72). Unless indicated otherwise, one-letter abbreviations in the lower case are used to indicate D-amino acids.

It is understood that names for various vaccines commonly indicate the molecular or disease target(s) for which a vaccine is intended to elicit an immune response, e.g., “influenza” or “flu” vaccines, “HPV” (human papilloma virus) vaccines, “polio” vaccines, “cholera” vaccines, “DPT” (diphtheria, pertussis, tetanus) vaccines, “MMR” (measles, mumps, rubella) vaccines, etc. Alternatively, reference to a vaccine may also be described with respect to the specificity of the immune response elicited by the vaccine, e.g., “anti-flu”, “anti-tetanus”, “anti-AIDS” vaccines. Accordingly, a vaccine composition described herein may properly be referred to as a “CETP vaccine” (emphasizing the target of vaccination) as well as an “anti-CETP vaccine” (emphasizing the specific immune response) without confusion to persons skilled in the art.

Nucleotides may be designated by of their conventional one-letter abbreviations, i.e., adenine (“A”), guanine (“G”), cytosine (“C”), thymine (“T”), and uracil (“U”).

“Individual” refers to a human or other mammal.

“Endogenous” as used and understood herein, refers to that which is produced by and present in an individual. For example, the CETP produced by and circulating in an individual is “endogenous” CETP. In contrast, the term “exogenous” refers to that which is from a source other than and outside of an individual. Although redundant, a phrase such as “the individual's own endogenous CETP” may be used to emphasize the fact that a vaccine composition as described herein elicits an autoimmune response directed to endogenous CETP present in the same individual who is administered the vaccine composition, and not to a foreign or exogenous protein or antigen. Within the context of discussing the source or origin of molecules, the term “homologous” is also understood by persons skilled in the art and may be used herein to refer to an endogenous molecule, e.g., endogenous CETP. Similarly, the term “heterologous” can be synonymous with the term “exogenous”. Thus, the meaning of the term “homologous” is understood by persons skilled in the art by the context in which it is used and is also readily distinguished from its other common usage in the art to describe the similarity between two or more nucleotide or amino acid sequences.

“Autoantibody” refers to an antibody produced by the immune system of an individual that recognizes (binds to) an endogenous molecule, e.g., endogenous CETP.

Vaccine peptides according to the invention are described herein as “antigenic” or “autoimmunogenic”, meaning that they elicit production of specific antibodies in an individual receiving the vaccine peptide which antibodies recognize or bind to the individual's endogenous CETP. Thus, the CETP vaccine peptides (also called “antigenic hybrid peptides”) of this invention are immunogenic moieties that have the capacity to stimulate the formation of endogenous antibodies which specifically bind endogenous CETP and/or inhibit endogenous CETP activity.

“Circulate” and “circulating” describe anything that travels or is otherwise transported through the vascular system of an individual. Thus, unless specifically indicated otherwise, “circulating CETP” is understood to refer to “endogenous CETP” present in the blood of an individual and as may be detected in the whole blood, serum, or plasma isolated from the vascular system of the individual.

A composition or method described herein as “comprising” one or more named elements or steps is open-ended meaning that the named elements or steps are essential, but other elements or steps may be included within the scope of the composition or method being described. To avoid prolixity, it is also understood that any composition or method described as “comprising” (or which “comprises”) one or more named elements or steps also describes the corresponding, more limited, composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method being described. It is also understood that any composition or method described herein as “comprising” or “consisting essentially of” one or more named elements or steps also describes the corresponding, more limited, and closed-ended composition or method “consisting of” (or which “consists of”) the named elements or steps to the exclusion of any other unnamed element or step. In any composition or method disclosed herein, known or disclosed equivalents of any named essential element or step may be substituted for that element or step.

As used herein, the terms “treatment” or “treating” refers to any regimen that alleviates one or more symptoms of a disease or disorder, that inhibits progression of a disease or disorder, that arrests progression or reverses progression (causes regression) of a disease or disorder, or that prevents onset of a disease or disorder. Treatment includes prophylaxis and includes but does not require cure of a disease or disorder.

Unless indicated otherwise, the meanings of other terms are the same as understood and used by persons skilled in the art.

The vaccine compositions described herein may be administered to an individual to elicit relatively high levels of antibodies that inhibit or reduce endogenous CETP activity in the individual, for altering the level of one or more lipoprotein-associated cholesterol molecules in the blood of the individual, for treating or preventing atherosclerosis in the individual, and combinations thereof.

### Universal or Broad Range Helper T Cell Epitopes

An antigenic hybrid polypeptide useful in vaccine compositions of the invention comprises a universal helper T cell epitope portion linked to a B cell epitope portion. The universal helper T cell portion comprises at least one broad range helper T cell epitope, which serves to activate helper T cells, which in turn stimulate antibody production from B cells.

Broad range helper T cell epitopes useful in the vaccine compositions described herein bind multiple class II MHC alleles expressed (as glycoproteins) on antigen-presenting cells. Such helper T cell epitopes have been referred to as “broad range”, “universal”, or “pan” MHC allele (e.g., pan DR epitopes). A number of naturally occurring broad range helper T cell epitopes are known that may be used in the CETP vaccine compositions described herein including, without limitation, peptides of tetanus toxoid (such as the tetanus toxoid fragment “TT 830-843”, see amino acids 2-15 of SEQ ID NO:1), diphtheria toxoid, pertussis vaccine, Bacile Calmette-Guerin (BCG), polio vaccine, measles vaccine, mumps vaccine, rubella vaccine, purified protein derivative of tuberculin, and like peptides.

An immunogenic carrier protein may also be used as the universal helper T cell epitope portion of the vaccine peptide. Such carrier proteins are selected because they have immunostimulatory properties presumably from the presence of several helper T cell epitope sites, and also include convenient binding site(s) for covalent attachment of one or more CETP B cell epitope portions. One such immunogenic carrier protein is keyhole limpet hemocyanin (KLH). KLH contains multiple lysine residues in its amino acid sequence, and each of these lysines is a potential site at which a B cell epitope peptide or a whole vaccine peptide as described herein could be linked (for example, using maleimide-activated KLH, Catalog No. 77106, Pierce Chemical Co., Rockford, Ill.). Other immunogenic carrier proteins useful in the present invention include heat shock proteins HSP70 and HSP65 from Mycobacterium tuberculosis.

A preferred naturally occurring broad range helper T cell epitope peptide is the TT 830-843 peptide that has the following sequence:
QYIKANSKFIGITE (nucleotides 2-15 of SEQ ID NO:1).
   Another broad range helper T cell epitope derived from tetanus toxin and useful herein is the peptide having the sequence:

| | |
|---|---|
| FNNFTVSFWLRVP KVSASHLE | (SEQ ID NO: 20) |

Whether a universal helper T cell epitope peptide recognizes (binds) multiple class II MHC allelic molecules may be readily determined by various assays, including T cell proliferation assays and peptide binding assays. A relatively large number of cell lines that express defined MHC alleles, e.g., alleles of the DR isotype, are available (e.g., from the American Type Culture Collection, Manassas, Va.) for use in such assays. In proliferation assays, a helper T cell epitope peptide is detected when it binds to an MHC allele expressed on antigen-presenting cells resulting in proliferation of co-cultured T cells (see, e.g., Panina-Bordignon et al., Eur. J. Immunol., 19: 2237-2242 (1989); Alexander et al., Immunity, 1: 751-761 (1994)). In binding assays, a purified peptide is tested for its ability to bind directly to one or more purified MHC allelic molecules. Such binding assays have been shown to accurately identify both naturally occurring and non-naturally occurring (i.e., synthetic) broad range helper T cell epitope peptides (see, e.g., Alexander et al., 1994, supra; U.S. Pat. No. 5,736,142).

An extensively characterized example of degenerate class II MHC binding is found in the case of the human DR isotype, in which multiple DR allelic molecules have been shown to bind (pan-DR binding) not only to previously known, naturally-occurring, broad range helper T cell epitopes (such as “TT 830-843”; amino acids 2-15 of SEQ ID NO:1) but also various families of non-naturally occurring (synthetic) peptides, wherein each of the non-naturally occurring T cell epitope peptides of a family share a common, non-naturally occurring amino acid sequence motif. Such broad range, non-naturally occurring helper T cell epitope peptides are also referred to as “pan DR peptides”, “pan DR epitopes”, or “PADRE” peptides (see, e.g., Alexander et al., Immunity, 1: 751-761 (1994); Del Guercio et al., Vaccine, 15: 441-448 (1997); Franke et al., Vaccine, 17: 1201-1205 (1999); U.S. Pat. No. 5,736,142; U.S. Pat. No. 6,413,935; U.S. Pat. No. 6,534,482). Non-naturally occurring, broad range helper T cell epitope peptides may include one or more D-amino acids (e.g., D-alanine) and/or modified amino acids (e.g., cyclohexylalanine, abbreviated as “Cha”; see below).

An example of a preferred non-naturally occurring pan DR epitope peptide that is useful as a broad range helper T cell epitope in an antigenic hybrid polypeptide according to the invention has the amino acid sequence:

| | |
|---|---|
| aKChaVAAWTLKAa, | (amino acids 1-12 of SEQ ID NO: 2) |

wherein “a” is D-alanine and “Cha” is cyclohexylalanine.

### B Cell Epitope Portion of CETP

The sequencing of cDNA encoding human CETP (see, Drayna et al., Nature, 327: 632-634 (1987) and rabbit CETP (see, Nagashima et al., J. Lipid Res., 29: 1643-1649 (1988) were followed by considerable work on the functional domains of CETP and the identification of various B cell epitopes throughout the amino acid sequence of the mammalian CETP molecules (see, e.g., Hesler et al., J. Biol. Chem., 263: 5020-5023 (1988); Swenson et al., J. Biol. Chem., 264: 14318-14326 (1989); Wang et al., J. Biol. Chem., 267: 17487-17490 (1992); Smith et al., Med. Sci. Res., 21: 911-912 (1993); Roy et al., J. Lipid Res., 37: 22-34 (1996)). B cell epitopes of a protein have classically been identified as portions of the protein that are recognized (bound) by antibodies elicited by an individual's immune system that recognizes the protein as undesired, foreign (non-self) material. It is now clear that when linked to a broad range helper T cell epitope, a B cell epitope of an individual's endogenous CETP can direct production of antibodies (autoantibodies) that bind to the individual's endogenous CETP and alter the profile of one or more lipoprotein-associated cholesterol molecules in a beneficial (anti-atherogenic) manner (see, e.g., U.S. Pat. No. 6,410,022; U.S. Pat. No. 6,555,113). Work conducted in the development of previously described vaccine compositions against endogenous CETP also demonstrates the usefulness and consistency of initially employing animal models of CETP-mediated cholesterol metabolism, such as rabbits, to test vaccine compositions for eventual use in humans (see, e.g., U.S. Pat. No. 6,410,022; U.S. Pat. No. 6,555,113; U.S. Pat. No. 6,284,533; U.S. Pat. No. 6,846,808; Davidson et al., 2003, supra). This appears to be so because the amino acid sequence of rabbit CETP is homologous, although not identical, to human CETP and also because the rabbit model for cholesterol-induced atherosclerosis is a reliable and controllable model of human atherosclerosis (see, e.g., U.S. Pat. No. 6,555,113).

In selecting the B cell epitope portion from a CETP sequence, it is advantageous to select a B cell epitope from a CETP of the same species as the individual to be vaccinated, in order to minimize immunogenicity (as distinguished from autoimmunogenicity) which might serve to neutralize the intended effect of the vaccine peptide. Thus, vaccine peptides intended for human use will preferably utilize B cell epitopes from human CETP.

Suitable B cell epitopes may be derived from any part of the CETP. Peptide segments of from six to twenty-one amino acids or longer are suitable (if they contain a CETP B cell epitope). Particular mention is made of the N-terminal twenty-one amino acids of human CETP and the C-terminal twenty-six amino acids of human CETP. Suitable B cell epitopes comprise six to twenty-one consecutive amino acids of the N-terminal twenty-one amino acids of human CETP or six to twenty-six consecutive amino acids of the C-terminal twenty-six amino acids of human CETP.

A preferred B cell epitope of human CETP that is useful in the antigenic hybrid peptides described herein has the following amino acid sequence:

| | |
|---|---|
| FGFPEHLLVDFLQSLS. | (amino acids 16-31 of |
| | SEQ ID NO: 1) |

Additional B cell epitope portions suitable for use in the present invention include a peptide having the sequence of the 21 amino-terminal amino acids of human CETP, i.e., CSKGTSHEAGIVCRITKPALL (SEQ ID NO:21) and a peptide having the sequence of amino acids 2-21 from the N-terminus of human CETP, i.e., SKGTSHEAGIVCRITKPALL (SEQ ID NO:22), wherein the N-terminal cysteine residue of human CETP has been removed.

### Antigenic Hybrid Polypeptides

An antigenic hybrid polypeptide useful in the vaccine compositions described herein comprises the two defined peptide portions mentioned above: a universal helper T cell epitope portion and a B cell epitope (of CETP) portion. The two peptide portions are linked to form a single antigenic (autoimmunogenic) hybrid polypeptide, however, as discussed below, the two portions may be linked directly to one another (e.g., via a peptide bond); linked via a linker molecule, which may or may not be a peptide; or linked indirectly to one another by linkage to a common carrier molecule. In addition, as discussed below, multiple (two or more) antigenic hybrid polypeptides may also be linked to another for use in a CETP vaccine composition of the invention.

In its most basic form, a single antigenic hybrid polypeptide useful in the CETP vaccine compositions of the invention has a universal helper T cell epitope portion linked directly via a peptide bond to a B cell epitope portion. In a preferred embodiment, a universal helper T cell epitope portion and a CETP B cell epitope portion are covalently linked end-to-end to form a continuous hybrid polypeptide. For example, a universal helper T cell epitope portion may be the amino terminal domain of an antigenic hybrid polypeptide and a B cell epitope portion may be the carboxyl terminal domain of the antigenic hybrid polypeptide (see, e.g., Example 1). Alternatively, the B cell epitope portion may be the amino terminal domain of the hybrid antigenic polypeptide and the helper T cell epitope portion may be the carboxyl terminal domain. Moreover, an antigenic hybrid polypeptide may have one or more helper T cell epitope portions linked to one or more B cell epitope portions.

A vaccine composition described herein may comprise one or more copies of the same or different antigenic hybrid polypeptides. For example, an antigenic hybrid polypeptide may have an amino terminal group that permits the polypeptide to be linked to other molecules, as when the polypeptide has an amino terminal cysteine residue (see, e.g., SEQ ID NO:1; Example). The sulfhydryl group of such an amino terminal cysteine provides a convenient means for linking two antigenic hybrid polypeptides together via a disulfide bond to form a dimer. Dimers may be homodimers of identical polypeptides or heterodimers of two different polypeptides. The presence of a sulfhydryl group for disulfide bond formation may also be used to link the antigenic hybrid polypeptide to any other molecule, substrate, or particle that is capable of forming a disulfide bond. In this way, another molecule, substrate, or particle that has multiple sulfhydryl groups available for disulfide bond formation may serve as a common carrier molecule to make vaccine compositions containing multiple (two or more) copies of antigenic hybrid polypeptides. Such disulfide bonds may be readily broken under proper reducing conditions and reformed under proper oxidizing conditions by methods available in the art without disrupting the essential peptide bonds of the individual antigenic hybrid polypeptides. As described in more detail below, the antigenic hybrid polypeptide may also be linked to the CpG oligonucleotide.

Of course, one or multiple copies of an antigenic hybrid polypeptide may also be attached to a common carrier molecule using linkages other than disulfide bonds. Examples of common carrier molecules that may be used in vaccine compositions described herein include, without limitation, serum proteins (e.g., serum albumin), “core” molecules (e.g., multiple antigenic peptide (MAP) arrangements; see, e.g., Tam et al., Proc. Natl. Acad. Sci. USA, 85: 5409-5413 (1988); Wang et al., Science, 254: 285-288 (1991); Marguerite et al., Mol. Immunol., 29: 793-800 (1992)), injectable resin particles, injectable polymeric particles, and the like, which have one or more functional groups available to form a bond with a single or multiple copies of an antigenic hybrid polypeptide described herein. In addition, by using the appropriate linkages or linker molecules (see, below), different species of antigenic hybrid polypeptides (e.g., having different amino acid sequences) may be attached to the same common carrier molecule.

Linking identical or different antigenic hybrid polypeptides to a common carrier molecule should not disrupt or significantly reduce the immunogenic properties of the antigenic hybrid polypeptides. Moreover, a preferred common carrier molecule does not introduce an epitope into a vaccine composition that does not contribute to the desired elicitation of a specific autoimmune response directed against the endogenous CETP of the individual who is administered the vaccine composition.

The carboxyl terminal amino acid residue of an antigenic hybrid polypeptide described herein may also be modified to block or reduce the reactivity of the free terminal carboxylic acid group, e.g., to prevent formation of esters, peptide bonds, and other reactions. Such blocking groups include forming an amide of the carboxylic acid group (see, Example 1). Other carboxylic acid groups that may be present in an antigenic hybrid polypeptide may also be blocked, again provided such blocking does not elicit an undesired immune reaction or significantly alter the capacity of the antigenic hybrid polypeptide to specifically elicit the production of antibodies in an individual to the individual's own endogenous CETP.

Linker molecules (“linkers”) may optionally be used to link a universal helper T cell epitope portion to a B cell epitope portion. Linkers may be peptides, which consist of one to multiple amino acids, or non-peptide molecules. Suitable linker molecules are those that link a helper T cell epitope portion to a B cell epitope portion and that do not make the resulting antigenic hybrid polypeptide toxic to the individual who is to receive the vaccine composition and that do not significantly interfere with or reduce the desired immunogenicity of the resulting antigenic hybrid polypeptide. Thus, preferred linker molecules do not introduce a further antigenic site that does not contribute to the specific and directed elicitation in a recipient individual of antibodies to the endogenous CETP of that individual. Preferred peptide linker molecules useful in the invention include glycine-rich peptide linkers that are T cell immunologically inert (see, e.g., U.S. Pat. No. 5,908,626), wherein more than half of the amino acid residues are glycine. Preferably, such glycine-rich peptide linkers consist of about 20 or fewer amino acids.

Linker molecules may also include non-peptide or partial peptide molecules. One or more universal helper T cell epitope portions may be linked to one or more B cell epitope portions using well known cross-linking molecules such as glutaraldehyde or EDC (Pierce, Rockford, Ill.). Bifunctional cross-linking molecules are linker molecules that possess two distinct reactive sites. For example, one of the reactive sites of a bifunctional linker molecule may be reacted with a functional group on a helper T cell epitope portion to form a covalent linkage and the other reactive site may be reacted with a functional group on a B cell epitope portion to form a covalent linkage, uniting the two portions to form an antigenic hybrid polypeptide. General methods for cross-linking molecules have been reviewed (see, e.g., Means and Feeney, Bioconjugate Chem., 1: 2-12 (1990)).

Homobifunctional cross-linker molecules have two reactive sites which are chemically the same. Examples of homobifunctional cross-linker molecules include, without limitation, glutaraldehyde; N,N′-bis(3-maleimido-propionyl-2-hydroxy-1,3-propanediol (a sulfhydryl-specific homobifunctional cross-linker); certain N-succinimide esters (e.g., discuccinimyidyl suberate, dithiobis(succinimidyl propionate), and soluble bis-sulfonic acid and salt thereof (see, e.g., Pierce Chemicals, Rockford, Ill.; Sigma-Aldrich Corp., St. Louis, Mo.). For this embodiment, the relative concentrations of universal helper T cell epitope portion peptides and B cell epitope portion peptides should be adjusted to maximize the number of universal helper T cell epitope and B cell epitope portions that are linked together and to minimize the linking of identical epitope portions to each other (i.e., to avoid formation of dimers of helper T cell epitope portions and dimers of B cell epitope portions).

Preferably, a bifunctional cross-linker molecule is a heterobifunctional linker molecule, meaning that the linker has at least two different reactive sites, each of which can be separately linked to a helper T cell or B cell epitope portion. Use of such heterobifunctional linkers permits chemically separate and stepwise addition (vectorial conjunction) of each of the reactive sites to a selected universal helper T cell portion or CETP B cell epitope portion. Heterobifunctional linker molecules useful in the invention include, without limitation, m-maleimidobenzoyl-N-hydroxysuccinimide ester (see, Green et al., Cell, 28: 477-487 (1982); Palker et al., Proc. Natl. Acad. Sci. (USA), 84: 2479-2483 (1987)); m-maleimido-benzoylsulfosuccinimide ester; γ-maleimidobutyric acid N-hydroxysuccinimide ester; and N-succinimidyl 3-(2-pyridyl-dithio)propionate (see, e.g., Carlos et al., Biochem. J., 173: 723-737 (1978); Sigma-Aldrich Corp., St. Louis, Mo.).

In another embodiment, the “antigenic hybrid polypeptide” of vaccine compositions described herein refers to the arrangement in which one or more universal helper T cell epitope portions and one or more B cell epitope portions are individually linked to a common carrier molecule, such as a serum protein (e.g., serum albumin), a core molecule (e.g., multiple antigenic peptide (MAP) arrangements, a resin particle, a polymeric particle, and the like. Linking individual helper T cell epitope and B cell epitope portions to a common carrier in this respect may be accomplished using any of a variety linkages, including, without limitation, multiple cross-linker molecules such as glutaraldehyde or other bifunctional linkers (see, above). In this type of linking, the relative concentrations of helper T cell epitope portion, B cell epitope portion, linker, and common carrier molecule should be adjusted to maximize the number of universal helper T cell epitope and CETP B cell epitope portions that are linked to the common carrier while minimizing both the direct linking of identical epitope portions to one another (homodimer formation) and of helper T cell epitope portions to different B cell epitope portions (heterodimer formation). Preferably, linking universal helper T cell epitope portions and CETP B cell epitope portions to a common carrier should not significantly disrupt or reduce the immunogenic properties of the universal helper T cell epitope portion or the B cell epitope portion and should not provide an additional undesired immunogenic domain that does not contribute to the directed elicitation of an immune response in an individual that is directed to the individual's endogenous CETP.

Polypeptides used in the vaccine compositions described herein may be produced by any of a variety of methods available for making polypeptides of known amino acid sequence. Such methods include using automated peptide synthesis using automated peptide synthesizers. Automated peptide synthesis is particularly useful in the case of novel peptides or peptides having D-amino acids, uncommon amino acids (e.g., norleucine), and modified amino acids (e.g., cyclohexylalanine). Polypeptides may also be produced using recombinant nucleic acid technology in which a polypeptide of specified amino acid sequence is properly expressed in a prokaryotic or eukaryotic cell from foreign nucleic acid, such as a DNA molecule encoding the polypeptide. Nucleic acid molecules encoding a specific polypeptide are readily produced by recombinant DNA manipulations, polymerase chain reaction (PCR) methods, and/or by automated DNA synthesis. The nucleic molecule encoding the desired polypeptide can then be inserted in an appropriate vector molecule (e.g., plasmid, bacteriophage, eukaryotic viral vector, mini-chromosome, etc.) for expression in an appropriate prokaryotic or eukaryotic host cell using standard methods. The expressed polypeptide may then be isolated from the expression system using any of a variety of standard methods to purify polypeptides, including but limited to, immunological methods, affinity methods, precipitation methods, and combinations thereof. Particularly useful for isolating polypeptides used in the vaccine compositions described herein are immunological methods that employ antibodies or fragments thereof that specifically bind a particular universal helper T cell epitope or CETP B cell epitope.

### Adjuvants

The discovery of the invention is based, in part, on the observation of significantly increased autoantibody titers elicited using vaccine compositions described herein that include a particular adjuvant in combination with a vaccine peptide as described above. Such adjuvants comprise immunostimulatory oligonucleotides such as an oligonucleotide having one or more CpG motifs that acts as an agonist of Toll-like receptor 9 (TLR9), such as CpG oligonucleotide adjuvants (Coley Pharmaceuticals, Inc., Wellesley, Mass.).

Additional adjuvant compositions may also be used, however the presence of an immunostimulatory oligonucleotide such as a CpG adjuvant is critical for the desirably high anti-endogenous CETP autoantibody titers that are induced according to the methods of the present invention. Such additional adjuvant(s) may be any adjuvant suitable for use in the mammalian subject to be vaccinated. For example, a preferred additional adjuvant used in vaccine compositions described herein is selected from the group consisting of an aluminum hydroxide adjuvant, an aluminum phosphate adjuvant, a calcium phosphate adjuvant, and combinations thereof. Most preferably, the additional (optional) adjuvant of a vaccine composition described herein is a colloidal suspension of aluminum hydroxide (also referred to as “alhydrogel”).

The essential adjuvant component of vaccine compositions described herein is the immunostimulatory oligonucleotide. The immunostimulatory oligonucleotides of the invention thus include at least one immunostimulatory motif. In a preferred embodiment the immunostimulatory motif is an “internal immunostimulatory motif”. The term “internal immunostimulatory motif” refers to the position of the motif sequence within a longer nucleic acid sequence, which is longer in length than the motif sequence by at least one nucleotide linked to both the 5′ and 3′ ends of the immunostimulatory motif sequence. The immunostimulatory oligonucleotide may be an RNA oligonucleotide (ORN) or a DNA oligonucleotide (ODN).

Preferably the immunostimulatory oligonucleotides include immunostimulatory motifs which are “CpG dinucleotides”. A CpG dinucleotide can be methylated or unmethylated. An immunostimulatory oligonucleotide containing at least one unmethylated CpG dinucleotide is an oligonucleotide which contains an unmethylated cytosine-guanine dinucleotide sequence (i.e., an unmethylated 5′ cytidine followed by 3′ guanosine and linked by a phosphate bond) and which activates the immune system. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

The methods of the invention may embrace the use of A class, B class C class, P class and T class CpG immunostimulatory nucleic acids. It has recently been described that there are different classes of CpG nucleic acids. One class is potent for activating B cells but is relatively weak in inducing IFN-α and NK cell activation; this class has been termed the B class. The B class CpG nucleic acids typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068. Another class is potent for inducing IFN-α and NK cell activation but is relatively weak at stimulating B cells; this class has been termed the A class. The A class CpG nucleic acids typically have stabilized poly-G sequences at 5′ and 3′ ends and a palindromic phosphodiester CpG dinucleotide-containing sequence of at least 6 nucleotides. See, for example, published patent application PCT/US00/26527 (WO 01/22990). Yet another class of CpG nucleic acids activates B cells and NK cells and induces IFN-α; this class has been termed the C-class. The C-class CpG nucleic acids, as first characterized, typically are fully stabilized, include a B class-type sequence and a GC-rich palindrome or near-palindrome. This class has been described in U.S. patent application Ser. No. 10/224,523, published under no. 2003-0148976 on Aug. 7, 2003.

“A class” CpG immunostimulatory nucleic acids have been described in U.S. Pat. No. 6,949,520 and published PCT application PCT/US00/26527 (WO 01/22990). These nucleic acids are characterized by the ability to induce high levels of interferon-alpha while having minimal effects on B cell activation. The A class CpG immunostimulatory nucleic acid typically are composed of a hexamer palindrome such as GACGTC, AGCGCT, or AACGTT described by Yamamoto and colleagues (Yamamoto S et al. J Immunol 148:4072-6 (1992)) surrounded by at least two G motifs on the 5′ side and at least 5 G motifs on the 3′ side. Additional nucleotides may separate the palindromic region from the G rich sections of the oligonucleotide. In some embodiments the central nucleotides have phosphodiester linkages and the G-rich nucleotides are linked by phosphorothioate bonds.

B class CpG immunostimulatory nucleic acids strongly activate human B cells but have minimal effects inducing interferon-α. B class CpG immunostimulatory nucleic acids have been described in U.S. Pat. Nos. 6,194,388 B1 and 6,239,116 B1, issued on Feb. 27, 2001 and May 29, 2001 respectively.

In one embodiment the invention provides a B class CpG oligonucleotide represented by at least the formula:

5′ X₁X₂CGX₃X₄ 3′

wherein X₁, X₂, X₃, and X₄ are nucleotides. In one embodiment X₂ is adenine, guanine, or thymine. In another embodiment X₃ is cytosine, adenine, or thymine.

In another embodiment the invention provides an isolated B class CpG oligonucleotide represented by at least the formula:

5′ N₁X₁X₂CGX₃X₄N₂ 3′

wherein X₁, X₂, X₃, and X₄ are nucleotides and N is any nucleotide and N₁ and N₂ are nucleic acid sequences composed of from about 0-25 N's each. In one embodiment X₁X₂ is a dinucleotide selected from the group consisting of: GpT, GpG, GpA, ApA, ApT, ApG, CpT, CpA, CpG, TpA, TpT, and TpG; and X₃X₄ is a dinucleotide selected from the group consisting of: TpT, ApT, TpG, ApG, CpG, TpC, ApC, CpC, TpA, ApA, and CpA. Preferably X₁X₂ is GpA or GpT and X₃X₄ is TpT. In other embodiments X₁ or X₂ or both are purines and X₃ or X₄ or both are pyrimidines or X₁X₂ is GpA and X₃ or X₄ or both are pyrimidines. In another preferred embodiment X₁X₂ is a dinucleotide selected from the group consisting of: TpA, ApA, ApC, ApG, and GpG. In yet another embodiment X₃X₄ is a dinucleotide selected from the group consisting of: TpT, TpA, TpG, ApA, ApG, GpA, and CpA. X₁X₂ in another embodiment is a dinucleotide selected from the group consisting of: TpT, TpG, ApT, GpC, CpC, CpT, TpC, GpT and CpG; X₃ is a nucleotide selected from the group consisting of A and T and X₄ is a nucleotide, but wherein when X₁X₂ is TpC, GpT, or CpG, X₃X₄ is not TpC, ApT or ApC.

In another preferred embodiment the CpG oligonucleotide has the sequence 5′ TCN₁TX₁X₂CGX₃X₄ 3′ (SEQ ID NO.:4). The CpG oligonucleotides of the invention in some embodiments include X₁X₂ selected from the group consisting of GpT, GpG, GpA and ApA and X₃X₄ is selected from the group consisting of TpT, CpT and TpC.

A preferred B class oligonucleotide is 5′TCGTCGTTTTGTCGTTTTGTCGTT3′ SEQ ID NO.:3.

The C class immunostimulatory nucleic acids contain at least two distinct motifs having unique stimulatory effects on cells of the immune system. Some of these ODN have both a traditional “stimulatory” CpG sequence and a “GC-rich” or “B-cell neutralizing” motif. These combination motif nucleic acids have immune stimulating effects that fall somewhere between those effects associated with traditional “class B” CpG ODN, which are strong inducers of B cell activation and dendritic cell (DC) activation, and those effects associated with a more recently described class of immune stimulatory nucleic acids (“class A” CpG ODN) which are strong inducers of IFN-α: and natural killer (NK) cell activation but relatively poor inducers of B-cell and DC activation. Krieg A M et al. (1995) Nature 374:546-9; Ballas Z K et al. (1996) J Immunol 157:1840-5; Yamamoto S et al. (1992) J Immunol 148:4072-6. While preferred class A CpG ODN have mixed or chimeric backbones, the B and C class ODN may have either stabilized, e.g., phosphorothioate, chimeric, or phosphodiester backbones, and in some preferred embodiments, they have semi-soft backbones.

The stimulatory domain or motif is defined by a formula: 5′ X₁DCGHX₂ 3′. D is a nucleotide other than C. C is cytosine. G is guanine. H is a nucleotide other than G.

X₁ and X₂ are any nucleic acid sequence 0 to 10 nucleotides long. X₁ may include a CG, in which case there is preferably a T immediately preceding this CG. In some embodiments DCG is TCG. X₁ is preferably from 0 to 6 nucleotides in length. In some embodiments X₂ does not contain any poly G or poly A motifs. In other embodiments the immunostimulatory nucleic acid has a poly-T sequence at the 5′ end or at the 3′ end. As used herein, “poly-A” or “poly-T” shall refer to a stretch of four or more consecutive A's or T's respectively, e.g., 5′ AAAA 3′ or 5′ TTTT 3′.

As used herein, “poly-G end” shall refer to a stretch of four or more consecutive G's, e.g., 5′ GGGG 3′, occurring at the 5′ end or the 3′ end of a nucleic acid. As used herein, “poly-G nucleic acid” shall refer to a nucleic acid having the formula 5′ X₁X₂GGGX₃X₄ 3′ wherein X₁, X₂, X₃, and X₄ are nucleotides and preferably at least one of X₃ and X₄ is a G.

Some preferred designs for the B cell stimulatory domain under this formula comprise TTTTTCG, TCG, TTCG, TTTCG, TTTTCG, TCGT, TTCGT, TTTCGT, TCGTCGT.

The second motif of the nucleic acid is referred to as either P or N and is positioned immediately 5′ to X₁ or immediately 3′ to X₂.

N is a B-cell neutralizing sequence that begins with a CGG trinucleotide and is at least 10 nucleotides long. A B-cell neutralizing motif includes at least one CpG sequence in which the CG is preceded by a C or followed by a G (Krieg A M et al. (1998) Proc Natl Acad Sci USA 95:12631-12636) or is a CG containing DNA sequence in which the C of the CG is methylated. As used herein, “CpG” shall refer to a 5′ cytosine (C) followed by a 3′ guanine (G) and linked by a phosphate bond. At least the C of the 5′ CG 3′ must be unmethylated. Neutralizing motifs are motifs which has some degree of immunostimulatory capability when present in an otherwise non-stimulatory motif, but, which when present in the context of other immunostimulatory motifs serve to reduce the immunostimulatory potential of the other motifs.

P is a GC-rich palindrome containing sequence at least 10 nucleotides long. As used herein, “palindrome” and, equivalently, “palindromic sequence” shall refer to an inverted repeat, i.e., a sequence such as ABCDEE′D′C′B′A′ in which A and A′, B and B′, etc., are bases capable of forming the usual Watson-Crick base pairs.

As used herein, “GC-rich palindrome” shall refer to a palindrome having a base composition of at least two-thirds G's and C's. In some embodiments the GC-rich domain is preferably 3′ to the “B cell stimulatory domain”. In the case of a 10-base long GC-rich palindrome, the palindrome thus contains at least 8 G's and C's. In the case of a 12-base long GC-rich palindrome, the palindrome also contains at least 8 G's and C's. In the case of a 14-mer GC-rich palindrome, at least ten bases of the palindrome are G's and C's. In some embodiments the GC-rich palindrome is made up exclusively of G's and C's.

In some embodiments the GC-rich palindrome has a base composition of at least 81 percent G's and C's. In the case of such a 10-base long GC-rich palindrome, the palindrome thus is made exclusively of G's and C's. In the case of such a 12-base long GC-rich palindrome, it is preferred that at least ten bases (83 percent) of the palindrome are G's and C's. In some preferred embodiments, a 12-base long GC-rich palindrome is made exclusively of G's and C's. In the case of a 14-mer GC-rich palindrome, at least twelve bases (86 percent) of the palindrome are G's and C's. In some preferred embodiments, a 14-base long GC-rich palindrome is made exclusively of G's and C's. The C's of a GC-rich palindrome can be unmethylated or they can be methylated.

In general this domain has at least 3 Cs and Gs, more preferably 4 of each, and most preferably 5 or more of each. The number of Cs and Gs in this domain need not be identical. It is preferred that the Cs and Gs are arranged so that they are able to form a self-complementary duplex, or palindrome, such as CCGCGCGG. This may be interrupted by As or Ts, but it is preferred that the self-complementarity is at least partially preserved as for example in the motifs CGACGTTCGTCG (SEQ ID NO:5) or CGGCGCCGTGCCG (SEQ ID NO:6). When complementarity is not preserved, it is preferred that the non-complementary base pairs be TG. In a preferred embodiment there are no more than 3 consecutive bases that are not part of the palindrome, preferably no more than 2, and most preferably only 1. In some embodiments the GC-rich palindrome includes at least one CGG trimer, at least one CCG trimer, or at least one CGCG tetramer. In other embodiments the GC-rich palindrome is not CCCCCCGGGGGG (SEQ ID NO:7) or GGGGGGCCCCCC (SEQ ID NO:8), CCCCCGGGGG (SEQ ID NO:9) or GGGGGCCCCC (SEQ ID NO:10).

At least one of the G's of the GC rich region may be substituted with an inosine (I). In some embodiments P includes more than one I.

In certain embodiments the immunostimulatory nucleic acid has one of the following formulas 5′ NX₁DCGHX₂ 3′, 5′ X₁DCGHX₂N 3′, 5′ PX₁DCGHX₂ 3′, 5′ X₁DCGHX₂P 3′, 5′ X₁DCGHX₂PX₃ 3′, 5′ X₁DCGHPX₃ 3′, 5′ DCGHX₂PX₃ 3′, 5′ TCGHX₂PX₃ 3′, 5′ DCGHPX₃ 3′, or 5′ DCGHP 3′.

In other aspects the invention provides immune stimulatory nucleic acids which are defined by a formula: 5′ N₁PyGN₂P 3′. N₁ is any sequence 1 to 6 nucleotides long. Py is a pyrimidine. G is guanine. N₂ is any sequence 0 to 30 nucleotides long. P is a GC-rich palindrome containing sequence at least 10 nucleotides long.

N₁ and N₂ may contain more than 50% pyrimidines, and more preferably more than 50% T. N₁ may include a CG, in which case there is preferably a T immediately preceding this CG. In some embodiments N₁PyG is TCG (such as ODN 5376, which has a 5′ TCGG), and most preferably a TCGN₂, where N₂ is not G.

N₁PyGN₂P may include one or more inosine (I) nucleotides. Either the C or the G in N₁ may be replaced by inosine, but the CpI is preferred to the IpG. For inosine substitutions such as IpG, the optimal activity may be achieved with the use of a “semi-soft” or chimeric backbone, where the linkage between the IG or the CI is phosphodiester. N, may include at least one CI, TCI, IG or TIG motif.

In certain embodiments N₁PyGN₂ is a sequence selected from the group consisting of TTTTTCG, TCG, TTCG, TTTCG, TTTTCG, TCGT, TTCGT, TTTCGT, and TCGTCGT.

Some non limiting examples of C-Class nucleic acids include:

| SEQ | |
|---|---|
| ID | |
| NO | Sequence |
| 11 | T*C_G*C_G*T*C_G*T*T*C_G*G*C*G*C_G*C*G*C*C*G |
| 12 | T*C_G*T*C_G*A*C_G*T*T*C_G*G*C*G*C_G*C*G*C*C*G |
| 13 | T*C_G*G*A*C_G*T*T*C_G*G*C*G*C_G*C*G*C*C*G |
| 14 | T*C_G*G*A*C_G*T*T*C_G*G*C*G*C*G*C*C*G |
| 15 | T*C_G*C_G*T*C_G*T*T*C_G*G*C*G*C*G*C*C*G |
| 16 | T*C_G*A*C_G*T*T*C_G*G*C*G*C_G*C*G*C*C*G |
| 17 | T*C_G*A*C_G*T*T*C_G*G*C*G*C*G*C*C*G |
| 18 | T*C_G*C_G*T*C_G*T*T*C_G*G*C*G*C*C*G |
| 19 | T*C_G*C_G*A*C_G*T*T*C_G*G*C*G*C_G*C*G*C*C*G |

The T class oligonucleotides induce secretion of lower levels of IFN-alpha and IFN-related cytokines and chemokines than B class or C class oligonucleotides, while retaining the ability to induce levels of IL-10 similar to B class oligonucleotides.

Another class, the P-Class oligonucleotides, have the ability in some instances to induce much higher levels of IFN-α secretion than the C-Class. The P-Class oligonucleotides have the ability to spontaneously self-assemble into concatamers either in vitro and/or in vivo. Without being bound by any particular theory for the method of action of these molecules, one potential hypothesis is that this property endows the P-Class oligonucleotides with the ability to more highly crosslink TLR9 inside certain immune cells, inducing a distinct pattern of immune activation compared to the previously described classes of CpG oligonucleotides.

In some embodiments of the invention the immunostimulatory oligonucleotide is an oligoribonucleotide (ORN). Immunostimulatory ORNs include for instance, those that stimulate TLR7/8 motifs. A TLR7/8 stimulating ORN may include for example a ribonucleotide sequence such as 5′-C/U-U-G/U-U-3′,5′-R-U-R-G-Y-3′, 5′-G-U-U-G-B-3′,5′-G-U-G-U-G/U-3′, or 5′-G/C-U-A/C-G-G-C-A-C-3′. C/U is cytosine (C) or uracil (U), G/U is guanine (G) or U, R is purine, Y is pyrimidine, B is U, G, or C, G/C is G or C, and A/C is adenine (A) or C. The 5′-C/U-U-G/U-U-3′ may be CUGU, CUUU, UUGU, or UUUU. In various embodiments 5′-R-U-R-G-Y-3′ is GUAGU, GUAGC, GUGGU, GUGGC, AUAGU, AUAGC, AUGGU, or AUGGC. In one embodiment the base sequence is GUAGUGU. In various embodiments 5′-G-U-U-G-B-3′ is GUUGU, GUUGG, or GUUGC. In various embodiments 5′-G-U-G-U-G/U-3′ is GUGUG or GUGUU. In one embodiment the base sequence is GUGUUUAC. In various other embodiments 5′-G/C-U-A/C-G-G-C-A-C-3′ is GUAGGCAC, GUCGGCAC, CUAGGCAC, or CUCGGCAC.

The immunostimulatory oligonucleotide molecules may have a chimeric backbone. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. Since boranophosphonate linkages have been reported to be stabilized relative to phosphodiester linkages, for purposes of the chimeric nature of the backbone, boranophosphonate linkages can be classified either as phosphodiester-like or as stabilized, depending on the context. For example, a chimeric backbone according to the instant invention could in one embodiment include at least one phosphodiester (phosphodiester or phosphodiester-like) linkage and at least one boranophosphonate (stabilized) linkage. In another embodiment a chimeric backbone according to the instant invention could include boranophosphonate (phosphodiester or phosphodiester-like) and phosphorothioate (stabilized) linkages. A “stabilized internucleotide linkage” shall mean an internucleotide linkage that is relatively resistant to in vivo degradation (e.g., via an exo- or endo-nuclease), compared to a phosphodiester internucleotide linkage. Preferred stabilized internucleotide linkages include, without limitation, phosphorothioate, phosphorodithioate, methylphosphonate, and methylphosphorothioate. Other stabilized internucleotide linkages include, without limitation: peptide, alkyl, dephospho, and others as described above.

Mixed backbone modified ODN may be synthesized using a commercially available DNA synthesizer and standard phosphoramidite chemistry. (F. E. Eckstein, “Oligonucleotides and Analogues—A Practical Approach” IRL Press Oxford, UK, 1991, and M. D. Matteucci and M. H. Caruthers, Tetrahedron Lett. 21, 719 (1980)). After coupling, PS linkages are introduced by sulfurization using the Beaucage reagent (R. P. Iyer, W. Egan, J. B. Regan and S. L. Beaucage, J. Am. Chem. Soc. 112, 1253 (1990)) (0.075 M in acetonitrile) or phenyl acetyl disulfide (PADS) followed by capping with acetic anhydride, 2,6-lutidine in tetrahydrofurane (1:1:8; v:v:v) and N-methylimidazole (16% in tetrahydrofurane). This capping step is performed after the sulfurization reaction to minimize formation of undesired phosphodiester (PO) linkages at positions where a phosphorothioate linkage should be located. In the case of the introduction of a phosphodiester linkage, e.g. at a CpG dinucleotide, the intermediate phosphorous-III is oxidized by treatment with a solution of iodine in water/pyridine. After cleavage from the solid support and final deprotection by treatment with concentrated ammonia (15 hrs at 50° C.), the ODN are analyzed by HPLC on a Gen-Pak Fax column (Millipore-Waters) using a NaCl-gradient (e.g. buffer A: 10 mM NaH₂PO₄ in acetonitrile/water=1:4/v:v pH 6.8; buffer B: 10 mM NaH₂PO₄, 1.5 M NaCl in acetonitrile/water=1:4/v:v; 5 to 60% B in 30 minutes at 1 ml/min) or by capillary gel electrophoresis. The ODN can be purified by HPLC or by FPLC on a Source High Performance column (Amersham Pharmacia). HPLC-homogeneous fractions are combined and desalted via a C18 column or by ultrafiltration. The ODN was analyzed by MALDI-TOF mass spectrometry to confirm the calculated mass.

In some embodiments the oligonucleotides may be soft or semi-soft oligonucleotides. A soft oligonucleotide is an immunostimulatory oligonucleotide having a partially stabilized backbone, in which phosphodiester or phosphodiester-like internucleotide linkages occur only within and immediately adjacent to at least one internal CG dinucleotide. The at least one internal CG dinucleotide itself has a phosphodiester or phosphodiester-like internucleotide linkage. A phosphodiester or phosphodiester-like internucleotide linkage occurring immediately adjacent to the at least one internal CG dinucleotide can be 5′, 3′, or both 5′ and 3′ to the at least one internal CG dinucleotide.

In particular, phosphodiester or phosphodiester-like internucleotide linkages involve “internal dinucleotides”. An internal dinucleotide in general shall mean any pair of adjacent nucleotides connected by an internucleotide linkage, in which neither nucleotide in the pair of nucleotides is a terminal nucleotide, i.e., neither nucleotide in the pair of nucleotides is a nucleotide defining the 5′ or 3′ end of the oligonucleotide. Thus a linear oligonucleotide that is n nucleotides long has a total of n−1 dinucleotides and only n−3 internal dinucleotides. Each internucleotide linkage in an internal dinucleotide is an internal internucleotide linkage. Thus a linear oligonucleotide that is n nucleotides long has a total of n−1 internucleotide linkages and only n−3 internal internucleotide linkages. The strategically placed phosphodiester or phosphodiester-like internucleotide linkages, therefore, refer to phosphodiester or phosphodiester-like internucleotide linkages positioned between any pair of nucleotides in the oligonucleotide sequence. In some embodiments the phosphodiester or phosphodiester-like internucleotide linkages are not positioned between either pair of nucleotides closest to the 5′ or 3′ end.

Preferably a phosphodiester or phosphodiester-like internucleotide linkage occurring immediately adjacent to the at least one internal CG dinucleotide is itself an internal internucleotide linkage. Thus for a sequence N₁ CG N₂, wherein N₁ and N₂ are each, independent of the other, any single nucleotide, the CG dinucleotide has a phosphodiester or phosphodiester-like internucleotide linkage, and in addition (a) N₁ and Y are linked by a phosphodiester or phosphodiester-like internucleotide linkage when N₁ is an internal nucleotide, (b) Z and N₂ are linked by a phosphodiester or phosphodiester-like internucleotide linkage when N₂ is an internal nucleotide, or (c) N₁ and Y are linked by a phosphodiester or phosphodiester-like internucleotide linkage when N₁ is an internal nucleotide and Z and N₂ are linked by a phosphodiester or phosphodiester-like internucleotide linkage when N₂ is an internal nucleotide.

Soft oligonucleotides according to the instant invention are believed to be relatively susceptible to nuclease cleavage compared to completely stabilized oligonucleotides. Without meaning to be bound to a particular theory or mechanism, it is believed that soft oligonucleotides of the invention are cleavable to fragments with reduced or no immunostimulatory activity relative to full-length soft oligonucleotides. Incorporation of at least one nuclease-sensitive internucleotide linkage, particularly near the middle of the oligonucleotide, is believed to provide an “off switch” which alters the pharmacokinetics of the oligonucleotide so as to reduce the duration of maximal immunostimulatory activity of the oligonucleotide. This can be of particular value in tissues and in clinical applications in which it is desirable to avoid injury related to chronic local inflammation or immunostimulation, e.g., the kidney.

A semi-soft oligonucleotide is an immunostimulatory oligonucleotide having a partially stabilized backbone, in which phosphodiester or phosphodiester-like internucleotide linkages occur only within at least one internal CG dinucleotide. Semi-soft oligonucleotides generally possess increased immunostimulatory potency relative to corresponding fully stabilized immunostimulatory oligonucleotides. Due to the greater potency of semi-soft oligonucleotides, semi-soft oligonucleotides may be used, in some instances, at lower effective concentations and have lower effective doses than conventional fully stabilized immunostimulatory oligonucleotides in order to achieve a desired biological effect.

It is believed that the foregoing properties of semi-soft oligonucleotides generally increase with increasing “dose” of phosphodiester or phosphodiester-like internucleotide linkages involving internal CG dinucleotides. Thus it is believed, for example, that generally for a given oligonucleotide sequence with five internal CG dinucleotides, an oligonucleotide with five internal phosphodiester or phosphodiester-like CG internucleotide linkages is more immunostimulatory than an oligonucleotide with four internal phosphodiester or phosphodiester-like CG internucleotide linkages, which in turn is more immunostimulatory than an oligonucleotide with three internal phosphodiester or phosphodiester-like CG internucleotide linkages, which in turn is more immunostimulatory than an oligonucleotide with two internal phosphodiester or phosphodiester-like CG internucleotide linkages, which in turn is more immunostimulatory than an oligonucleotide with one internal phosphodiester or phosphodiester-like CG internucleotide linkage. Importantly, inclusion of even one internal phosphodiester or phosphodiester-like CG internucleotide linkage is believed to be advantageous over no internal phosphodiester or phosphodiester-like CG internucleotide linkage. In addition to the number of phosphodiester or phosphodiester-like internucleotide linkages, the position along the length of the oligonucleotide can also affect potency.

The soft and semi-soft oligonucleotides will generally include, in addition to the phosphodiester or phosphodiester-like internucleotide linkages at preferred internal positions, 5′ and 3′ ends that are resistant to degradation. Such degradation-resistant ends can involve any suitable modification that results in an increased resistance against exonuclease digestion over corresponding unmodified ends. For instance, the 5′ and 3′ ends can be stabilized by the inclusion there of at least one phosphate modification of the backbone. In a preferred embodiment, the at least one phosphate modification of the backbone at each end is independently a phosphorothioate, phosphorodithioate, methylphosphonate, or methylphosphorothioate internucleotide linkage. In another embodiment, the degradation-resistant end includes one or more nucleotide units connected by peptide or amide linkages at the 3′ end.

A phosphodiester internucleotide linkage is the type of linkage characteristic of nucleic acids found in nature. The phosphodiester internucleotide linkage includes a phosphorus atom flanked by two bridging oxygen atoms and bound also by two additional oxygen atoms, one charged and the other uncharged. Phosphodiester internucleotide linkage is particularly preferred when it is important to reduce the tissue half-life of the oligonucleotide.

A phosphodiester-like internucleotide linkage is a phosphorus-containing bridging group that is chemically and/or diastereomerically similar to phosphodiester. Measures of similarity to phosphodiester include susceptibility to nuclease digestion and ability to activate RNAse H. Thus for example phosphodiester, but not phosphorothioate, oligonucleotides are susceptible to nuclease digestion, while both phosphodiester and phosphorothioate oligonucleotides activate RNAse H. In a preferred embodiment the phosphodiester-like internucleotide linkage is boranophosphate (or equivalently, boranophosphonate) linkage. U.S. Pat. No. 5,177,198; U.S. Pat. No. 5,859,231; U.S. Pat. No. 6,160,109; U.S. Pat. No. 6,207,819; Sergueev et al., (1998) J Am Chem Soc 120:9417-27. In another preferred embodiment the phosphodiester-like internucleotide linkage is diasteromerically pure Rp phosphorothioate. It is believed that diasteromerically pure Rp phosphorothioate is more susceptible to nuclease digestion and is better at activating RNAse H than mixed or diastereomerically pure Sp phosphorothioate. Stereoisomers of CpG oligonucleotides are the subject of co-pending U.S. patent application Ser. No. 09/361,575 filed Jul. 27, 1999, and published PCT application PCT/US99/17100 (WO 00/06588). It is to be noted that for purposes of the instant invention, the term “phosphodiester-like internucleotide linkage” specifically excludes phosphorodithioate and methylphosphonate internucleotide linkages.

As described above the soft and semi-soft oligonucleotides of the invention may have phosphodiester like linkages between C and G. One example of a phosphodiester-like linkage is a phosphorothioate linkage in an Rp conformation. Oligonucleotide p-chirality can have apparently opposite effects on the immune activity of a CpG oligonucleotide, depending upon the time point at which activity is measured. At an early time point of 40 minutes, the Rₚ but not the S_{P} stereoisomer of phosphorothioate CpG oligonucleotide induces JNK phosphorylation in mouse spleen cells. In contrast, when assayed at a late time point of 44 hr, the S_{P} but not the Rₚ stereoisomer is active in stimulating spleen cell proliferation. This difference in the kinetics and bioactivity of the Rₚ and S_{P} stereoisomers does not result from any difference in cell uptake, but rather most likely is due to two opposing biologic roles of the p-chirality. First, the enhanced activity of the Rp stereoisomer compared to the Sp for stimulating immune cells at early time points indicates that the Rp may be more effective at interacting with the CpG receptor, TLR9, or inducing the downstream signaling pathways. On the other hand, the faster degradation of the Rp PS-oligonucleotides compared to the Sp results in a much shorter duration of signaling, so that the Sp PS-oligonucleotides appear to be more biologically active when tested at later time points.

The size (i.e., the number of nucleotide residues along the length of the oligonucleotide) of the immunostimulatory oligonucleotide may also contribute to the stimulatory activity of the oligonucleotide. For facilitating uptake into cells immunostimulatory oligonucleotides preferably have a minimum length of 6 nucleotide residues. Oligonucleotides of any size greater than 6 nucleotides (even many kb long) are capable of inducing an immune response according to the invention if sufficient immunostimulatory motifs are present, since larger oligonucleotides are degraded inside of cells. It is believed by the instant inventors that semi-soft oligonucleotides as short as 4 nucleotides can also be immunostimulatory if they can be delivered to the interior of the cell. In certain preferred embodiments according to the instant invention, the immunostimulatory oligonucleotides are between 4 and 100 nucleotides long. In typical embodiments the immunostimulatory oligonucleotides are between 6 and 40 nucleotides long. In certain preferred embodiments according to the instant invention, the immunostimulatory oligonucleotides are between 6 and 25 nucleotides long.

The terms “nucleic acid” and “oligonucleotide” also encompass nucleic acids or oligonucleotides with substitutions or modifications, such as in the bases and/or sugars. For example, they include oligonucleotides having backbone sugars that are covalently attached to low molecular weight organic groups other than a hydroxyl group at the 2′ position and other than a phosphate group or hydroxy group at the 5′ position. Thus modified oligonucleotides may include a 2′-O-alkylated ribose group. In addition, modified oligonucleotides may include sugars such as arabinose or 2′-fluoroarabinose instead of ribose. Thus the oligonucleotides may be heterogeneous in backbone composition thereby containing any possible combination of polymer units linked together such as peptide-nucleic acids (which have an amino acid backbone with nucleic acid bases).

Oligonucleotides also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, thymine, 5-methylcytosine, 5-hydroxycytosine, 5-fluorocytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art.

The immunostimulatory oligonucleotides of the instant invention can encompass various chemical modifications and substitutions, in comparison to natural RNA and DNA, involving a phosphodiester internucleotide bridge, a β-D-ribose unit and/or a natural nucleotide base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al. (1996) Annu Rev Pharmacol Toxicol 36:107-129; and Hunziker J et al. (1995) Mod Synth Methods 7:331-417. An oligonucleotide according to the invention may have one or more modifications, wherein each modification is located at a particular phosphodiester internucleotide bridge and/or at a particular β-D-ribose unit and/or at a particular natural nucleotide base position in comparison to an oligonucleotide of the same sequence which is composed of natural DNA or RNA.

For example, the invention relates to an oligonucleotide which may comprise one or more modifications and wherein each modification is independently selected from:
a) the replacement of a phosphodiester internucleotide bridge located at the 3′ and/or the 5′ end of a nucleotide by a modified internucleotide bridge,
b) the replacement of phosphodiester bridge located at the 3′ and/or the 5′ end of a nucleotide by a dephospho bridge,
c) the replacement of a sugar phosphate unit from the sugar phosphate backbone by another unit,
d) the replacement of a β-D-ribose unit by a modified sugar unit, and
e) the replacement of a natural nucleotide base by a modified nucleotide base.

More detailed examples for the chemical modification of an oligonucleotide are as follows.

A phosphodiester internucleotide bridge located at the 3′ and/or the 5′ end of a nucleotide can be replaced by a modified internucleotide bridge, wherein the modified internucleotide bridge is for example selected from phosphorothioate, phosphorodithioate, NR¹R²-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)—O-alkyl]ester, (C₁-C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleotide by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethylhydrazo, dimethylenesulfone and/or silyl groups.

A sugar phosphate unit (i.e., a β-D-ribose and phosphodiester internucleotide bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Nucleic Acids Res 17:6129-41), that is, e.g., the replacement by a morpholino-derivative unit; or to build up a polyamide nucleic acid (“PNA”; as described for example, in Nielsen P E et al. (1994) Bioconjug Chem 5:3-7), that is, e.g., the replacement by a PNA backbone unit, e.g., by 2-aminoethylglycine.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′—NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some preferred embodiments the sugar is 2′-O-methylribose, particularly for one or both nucleotides linked by a phosphodiester or phosphodiester-like internucleotide linkage.

Oligonucleotides also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, and thymine, and other naturally and non-naturally occurring nucleobases, substituted and unsubstituted aromatic moieties.

A modified base is any base which is chemically distinct from the naturally occurring bases typically found in DNA and RNA such as T, C, G, A, and U, but which share basic chemical structures with these naturally occurring bases. The modified nucleotide base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleotides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleotide bases. This list is meant to be exemplary and is not to be interpreted to be limiting.

In particular formulas described herein a set of modified bases is defined. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g. 3-nitropyrrole, P-base), an aromatic ring system (e.g. fluorobenzene or difluorobenzene) or a hydrogen atom (dSpacer).

A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. In another embodiment of the invention, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

The oligonucleotides may be linked to one another, to carriers, or to the antigen using a variety of orientations and linkers, nucleotidic and non-nucleotidic. The linkers described here fall within the broad definition of linkers described above. In one possible orientation, the oligonucleotides may have one or more accessible 5′ ends. It is possible to create modified oligonucleotides having two such 5′ ends. This may be achieved, for instance by attaching two oligonucleotides through a 3′-3′ linkage to generate an oligonucleotide having one or two accessible 5′ ends. The 3′3′-linkage may be a phosphodiester, phosphorothioate or any other modified internucleotide bridge. Methods for accomplishing such linkages are known in the art. For instance, such linkages have been described in Seliger, H. et al., Oligonucleotide analogs with terminal 3′-3′- and 5′-5′-internucleotidic linkages as antisense inhibitors of viral gene expression, Nucleotides & Nucleotides (1991), 10(1-3), 469-77 and Jiang, et al., Pseudo-cyclic oligonucleotides: in vitro and in vivo properties, Bioorganic & Medicinal Chemistry (1999), 7(12), 2727-2735.

Additionally, 3′3′-linked oligonucleotides where the linkage between the 3′-terminal nucleotides is not a phosphodiester, phosphorothioate or other modified bridge, can be prepared using an additional spacer, such as tri- or tetra-ethylenglycol phosphate moiety (Durand, M. et al, Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat. No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Nucleic Acids Research (1994), 22(11), 2022-7) using standard phosphoramidite chemistry. The non-nucleotidic linkers can be incorporated once or multiple times, or combined with each other allowing for any desirable distance between the 3′-ends of the two ODNs to be linked.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann, E. and Peyman, A., Chem. Rev. 90:544, 1990; Goodchild, J., Bioconjugate Chem. 1: 165, 1990).

Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

DNA is a polymer of deoxyribonucleotides joined through 3′-5′ phosphodiester linkages. Units of the polymer of the invention can also be joined through 3′-5′ phosphodiester linkages. However, the invention also encompasses polymers having unusual internucleotide linkages, including specifically 5′-5′,3′-3′,2′-2′,2′-3′, and 2′-5′ internucleotide linkages. In one embodiment such unusual linkages are excluded from the immunostimulatory DNA motif, even though one or more of such linkages may occur elsewhere within the polymer. For polymers having free ends, inclusion of one 3′-3′ internucleotide linkage can result in a polymer having two free 5′ ends. Conversely, for polymers having free ends, inclusion of one 5′-5′ internucleotide linkage can result in a polymer having two free 3′ ends.

An immunostimulatory composition of this invention can contain two or more immunostimulatory DNA or RNA motifs which can be linked through a branching unit. The internucleotide linkages can be 3′-5′,5′-5′,3′-3′,2′-2′,2′-3′, or 2′-5′ linkages. Thereby, the nomenclature 2′-5′ is chosen according to the carbon atom of ribose or deoxyribose. However, if unnatural sugar moieties are employed, such as ring-expanded sugar analogs (e.g., hexanose, cylohexene or pyranose) or bi- or tricyclic sugar analogs, then this nomenclature changes according to the nomenclature of the monomer. The unusual internucleotide linkage can be a phosphodiester linkage, but it can alternatively be modified as phosphorothioate or any other modified linkage as described herein. Formula I shows a general structure for branched DNA or RNA oligomers and modified oligoribonucleotide analogs of the invention via a nucleotidic branching unit. Thereby Nu₁, Nu₂, and Nu₃ can be linked through 3′-5′,5′-5′,3′-3′,2′-2′,2′-3′, or 2′-5′-linkages. Branching of DNA oligomers can also involve the use of non-nucleotidic linkers and abasic spacers. In one embodiment, Nu₁, Nu₂, and Nu₃ represent identical or different immunostimulatory DNA or RNA motifs.

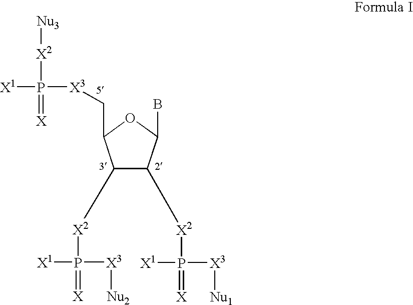

The modified oligoribonucleotide analog may contain a doubler or trebler unit (Glen Research, Sterling, Va.), in particular those modified oligodeoxyribonucleotide analogs with a 3′-3′ linkage. A doubler unit in one embodiment can be based on 1,3-bis-[5-(4,4′-dimethoxytrityloxy)pentylamido]propyl-2-[(2-cyanoethyl)-(N,N-diisopropyl)]-phosphoramidite. A trebler unit in one embodiment can be based on incorporation of Tris-2,2,2-[3-(4,4′-dimethoxytrityloxy)propyloxymethyl]ethyl-[(2-cyanoethyl)-(N,N-diisopropyl)]-phosphoramidite. Branching of the modified oligoribonucleotide analogs by multiple doubler, trebler, or other multiplier units leads to dendrimers which are a further embodiment of this invention. Branched modified oligoribonucleotide analogs may lead to crosslinking of receptors particularly for combinations of immunostimulatory RNA and DNA such as TLR3, TLR7, TLR8, and TLR9 with distinct immune effects compared to non-branched forms of the analogs. In addition, the synthesis of branched or otherwise multimeric analogs may stabilize DNA against degradation and may enable weak or partially effective DNA sequences to exert a therapeutically useful level of immune activity. The modified oligodeoxyribonucleotide analogs may also contain linker units resulting from peptide modifying reagents or oligonucleotide modifying reagents (Glen Research). Furthermore, the modified oligodeoxyribonucleotide analogs may contain one or more natural or unnatural amino acid residues which are connected to the polymer by peptide (amide) linkages.

Another possibility for linking oligonucleotides is via crosslinking of the heterocyclic bases (Verma and Eckstein; Annu. Rev. Biochem. (1998) 67: 99-134; page 124). A linkage between the sugar moiety of one sequence part with the heterocyclic base of another sequence part (Iyer et al. Curr. Opin. Mol. Therapeutics (1999) 1: 344-358; page 352) may also be used.

The different oligonucleotides are synthesized by established methods and can be linked together on-line during solid-phase synthesis. Alternatively, they may be linked together post-synthesis of the individual partial sequences.

The 3′-5′,5′-5′,3′-3′,2′-2′,2′-3′, and 2′-5′ internucleotide linkages can be direct or indirect. Direct linkages in this context refers to a phosphate or modified phosphate linkage as disclosed herein, without an intervening linker moiety. An intervening linker moiety is an organic moiety distinct from a phosphate or modified phosphate linkage as disclosed herein, which can include, for example, polyethylene glycol, triethylene glycol, hexaethylene glycol, dSpacer (i.e., an abasic deoxynucleotide), doubler unit, or trebler unit.

In principle, linkages between different parts of an oligonucleotide or between different oligonucleotides, respectively, can occur via all parts of the molecule, as long as this does not negatively interfere with the recognition by its receptor. According to the nature of the oligonucleotide, the linkage can involve the sugar moiety (Su), the heterocyclic nucleobase (Ba) or the phosphate backbone (Ph). Thus, linkages of the type Su-Su, Su-Ph, Su-Ba, Ba-Ba, Ba-Su, Ba-Ph, Ph-Ph, Ph-Su, and Ph-Ba are possible. If the oligonucleotides are further modified by certain non-nucleotidic substituents, the linkage can also occur via the modified parts of the oligonucleotides. These modifications also include modified oligonucleotides, e.g. PNA, LNA, or Morpholino Oligonucleotide analogs.

The linkages between oligonucleotides are preferably composed of C, H, N, O, S, B, P, and Halogen, containing 3 to 300 atoms. An example with 3 atoms is an acetal linkage (ODN1-3′-O—CH₂—O-3′-ODN2) connecting e.g. the 3′-hydroxy group of one nucleotide to the 3′-hydroxy group of a second oligonucleotide. An example with about 300 atoms is PEG-40 (tetraconta polyethyleneglycol). Preferred linkages are phosphodiester, phosphorothioate, methylphosphonate, phosphoramidate, boranophosphonate, amide, ether, thioether, acetal, thioacetal, urea, thiourea, sulfonamide, Schiff Base and disulfide linkages. It is also possible to use the Solulink BioConjugation System i.e., (www.trilinkbiotech.com).

If the oligonucleotide is composed of two or more sequence parts, these parts can be identical or different. Thus, in an oligonucleotide with a 3′3′-linkage, the sequences can be identical 5′-ODN1-3′3′-ODN1-5′ or different 5′-ODN1-3′3′-ODN2-5′. Furthermore, the chemical modification of the various oligonucleotide parts as well as the linker connecting them may be different. Since the uptake of short oligonucleotides appears to be less efficient than that of long oligonucleotides, linking of two or more short sequences results in improved immune stimulation. The length of the short oligonucleotides is preferably 2-20 nucleotides, more preferably 3-16 nucleotides, but most preferably 5-10 nucleotides. Preferred are linked oligonucleotides which have two or more unlinked 5′-ends.

In one embodiment the immunostimulatory ODN of the invention is advantageously combined with a cationic lipid. In one embodiment the cationic lipid is DOTAP (N-[1-(2,3-dioleoyloxy)propy-1]-N,N,N-trimethylammonium methyl-sulfate). Other agents with similar properties including trafficking to the endosomal compartment can be used in place of or in addition to DOTAP. Other lipid formulations include, for example, as EFFECTENE™ (a non-liposomal lipid with a special DNA condensing enhancer) and SUPERFECT™ (a novel acting dendrimeric technology). Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnol 3:235-241.

In one embodiment the immunostimulatory ODN of the invention are in the form of covalently closed, dumbbell-shaped molecules with both primary and secondary structure. In one embodiment such cyclic oligoribonucleotides include two single-stranded loops connected by an intervening double-stranded segment. In one embodiment at least one single-stranded loop includes an immunostimulatory DNA motif of the invention. Other covalently closed, dumbbell-shaped molecules of the invention include chimeric DNA:RNA molecules in which, for example, the double-stranded segment is at least partially DNA (e.g., either homodimeric dsDNA or heterodimeric DNA:RNA) and at least one single-stranded loop includes an immunostimulatory DNA motif of the invention. Alternatively, the double stranded segment of the chimeric molecule is DNA.

In certain embodiments the immunostimulatory ODN is isolated. An isolated molecule is a molecule that is substantially pure and is free of other substances with which it is ordinarily found in nature or in in vivo systems to an extent practical and appropriate for its intended use. In particular, the immunostimulatory ODN are sufficiently pure and are sufficiently free from other biological constituents of cells so as to be useful in, for example, producing pharmaceutical preparations. Because an isolated immunostimulatory ODN of the invention may be admixed with a pharmaceutically acceptable carrier in a pharmaceutical preparation, the immunostimulatory ODN may comprise only a small percentage by weight of the preparation. The immunostimulatory ODN is nonetheless substantially pure in that it has been substantially separated from the substances with which it may be associated in living systems.

The CpG oligonucleotides signal through TLR9. The RNA oligonucleotides are believed to signal through TLR7 and/or TLR8. As used herein, the term “TLR signaling” refers to any aspect of intracellular signaling associated with signaling through a TLR. As used herein, the term “TLR-mediated immune response” refers to the immune response that is associated with TLR signaling.

A TLR9-mediated immune response is a response associated with TLR9 signaling. This response is further characterized at least by the production/secretion of IFN-γ and IL-12, albeit at levels lower than are achieved via a TLR8-mediated immune response. As used herein, the term “TLR9 ligand” or “TLR9 agonist” refers to any agent that is capable of increasing TLR9 signaling (i.e., an agonist of TLR9). TLR9 ligands specifically include, without limitation, immunostimulatory oligonucleotides and in particular CpG oligonucleotides.

A TLR7-mediated immune response is a response associated with TLR7 signaling. TLR7-mediated immune response is generally characterized by the induction of IFN-α and IFN-inducible cytokines such as IP-10 and I-TAC. The levels of cytokines IL-1 α/β, IL-6, IL-8, MIP-1α/β and MIP-3α/β induced in a TLR7-mediated immune response are less than those induced in a TLR8-mediated immune response.

A TLR8-mediated immune response is a response associated with TLR8 signaling. This response is further characterized by the induction of pro-inflammatory cytokines such as IFN-γ, IL-12p40/70, TNF-α, IL-1α/β, IL-6, IL-8, MIP-1 α/β and MIP-3 α/β.

For use in the method of vaccinating a subject, the composition includes an antigen and a CpG oligonucleotide. The antigen can be separate from or covalently linked to a CpG oligonucleotide of the invention. In one embodiment the composition does not itself include the antigen. In this embodiment the antigen can be administered to the subject either separately from the CpG oligonucleotide, or together with the CpG oligonucleotide. Administration that is separate includes separate in time, separate in location or route of administration, or separate both in time and in location or route of administration. When the CpG oligonucleotide and the antigen are administered separate in time, the antigen can be administered before or after the CpG oligonucleotide. In one embodiment the antigen is administered 48 hours to 4 weeks after administration of the CpG oligonucleotide. The method also contemplates the administration of one or more booster doses of antigen alone, CpG oligonucleotide alone, or antigen and CpG oligonucleotide, following an initial administration of antigen and CpG oligonucleotide.

The CpG oligonucleotide can be linked to the antigen in a variety of ways. The link can be made at the 3′ or 5′ end of the CpG oligonucleotide, or to a suitably modified base at an internal position in the CpG oligonucleotide. If the antigen contains a suitable reactive group (e.g., an N-hydroxysuccinimide ester) it can be reacted directly with the N₄ amino group of cytosine residues. Depending on the number and location of cytosine residues in the CpG oligonucleotide, specific labeling at one or more residues can be achieved.

Alternatively, modified oligonucleosides, such as are known in the art, can be incorporated at either terminus, or at internal positions in the CpG oligonucleotide. These can contain blocked functional groups which, when deblocked, are reactive with a variety of functional groups which can be present on, or attached to, an antigen of interest.

The antigen can be attached to the 3′-end of the CpG oligonucleotide through solid support chemistry. For example, the CpG oligonucleotide portion can be added to a polypeptide portion that has been pre-synthesized on a support (Haralambidis et al., Nucleic Acids Res. (1990) 18:493-99; Haralambidis et al., Nucleic Acids Res. (1990) 18:501-505). Alternatively, the CpG oligonucleotide can be synthesized such that it is connected to a solid support through a cleavable linker extending from the 3′-end. Upon chemical cleavage of the CpG oligonucleotide from the support, a terminal thiol group is left at the 3′-end of the CpG oligonucleotide (Zuckermann et al., Nucleic Acids Res. (1987) 15:5305-5321; Corey et al., (1987) Science 238:1401-1403), or a terminal amine group is left at the 3′-end of the CpG oligonucleotide (Nelson et al., Nucleic Acids Res. (1989) 17:1781-94). Conjugation of the amino-modified CpG oligonucleotide to amino groups of the antigen can be performed as described in Benoit et al., Neuromethods (1987) 6:43-72. Conjugation of the thiol-modified CpG oligonucleotide to carboxyl groups of the antigen can be performed as described in Sinah et al., Oligonucleotide Analogues: A Practical Approach (1991) IRL Press.

The antigen can be attached to the 5′-end of the CpG oligonucleotide through an amine, thiol, or carboxyl group that has been incorporated into the CpG oligonucleotide during its synthesis. Preferably, while the CpG oligonucleotide is fixed to the solid support, a linking group comprising a protected amine, thiol, or carboxyl at one end, and a phosphoramidite at the other, is covalently attached to the 5′-hydroxyl (Agrawal et al., Nucleic Acids Res. (1986) 14:6227-6245; Connolly, Nucleic Acids Res. (1985) 13:4485-4502; Coull et al., Tetrahedron Lett. (1986) 27:3991-3994; Kremsky et al., Nucleic Acids Res. (1987) 15:2891-2909; Connolly, Nucleic Acids Res. (1987) 15:3131-3139; Bischoff et al., Anal. Biochem. (1987) 164:336-344; Blanks et al., Nucleic Acids Res. (1988) 16:10283-10299; U.S. Pat. Nos. 4,849,513; 5,015,733; 5,118,800; and 5,118,802). Subsequent to deprotection, the latent amine, thiol, and carboxyl functionalities can be used to covalently attach the CpG oligonucleotide to an antigen (Benoit et al., Neuromethods (1987) 6:43-72; Sinah et al., Oligonucleotide Analogues: A Practical Approach (1991) IRL Press).

An antigen can be attached to a modified cytosine or uracil at any position in the CpG oligonucleotide. The incorporation of a “linker arm,” possessing a latent reactive functionality, such as an amine or carboxyl group, at C-5 of the modified base provides a handle for the peptide linkage (Ruth, 4th Annual Congress for Recombinant DNA Research, p. 123).

The linkage of the CpG oligonucleotide to an antigen can also be formed through a high-affinity, non-covalent interaction such as a biotin-streptavidin complex. A biotinyl group can be attached, for example, to a modified base of a CpG oligonucleotide (Roget et al., Nucleic Acids Res. (1989) 1.7:7643-7651). Incorporation of a streptavidin moiety into the antigen allows formation of a non-covalently bound complex of the streptavidin conjugated antigen and the biotinylated CpG oligonucleotide.

The linkage of the CpG oligonucleotide to a lipid can be formed using standard methods. These methods include, but are not limited to, the synthesis of oligonucleotide-phospholipid conjugates (Yanagawa et al., Nucleic Acids Symp. Ser. (1988) 19:189-92), oligonucleotide-fatty acid conjugates (Grabarek et al., Anal. Biochem. (1990) 185:131-35; Staros et al., Anal. Biochem. (1986) 156:220-22), and oligonucleotide-sterol conjugates (Boujrad et al., Proc. Natl. Acad. Sci. USA (1993) 90:5728-31).

Additional methods for the attachment of peptides and other molecules to oligonucleotides can be found in C. Kessler: Nonradioactive labeling methods for nucleic acids in L. J. Kricka (ed.) “Nonisotopic DNA Probe Techniques,” Academic Press 1992 and in Geoghegan and Stroh, Bioconjug. Chem., 3:138-146, 1992.

Use and formulation of CETP vaccine compositions

CETP vaccine compositions described herein are designed to elicit production of anti-CETP antibodies in an individual that recognize the individual's own endogenous CETP at levels significantly higher than have been obtained with previously described vaccine compositions against CETP. A CETP vaccine composition as described herein may thus be used to improve any method of using previously described CETP vaccines including, but not limited to, a method of treating or preventing atherosclerosis in an individual; a method of increasing the level of HDL-C in the blood of an individual; a method of increasing the ratio of HDL-C to LDL-C, VLDL-C, or total cholesterol in the blood of an individual; a method of decreasing the level of LDL-C or VLDL-C in the blood of an individual; a method of inhibiting endogenous CETP activity in the blood of an individual; a method of clearing CETP molecules from the blood of an individual; and combinations thereof. Controlling the circulating level in an individual of one or more forms of lipoprotein-associated cholesterol, e.g., HDL-C, LDL-C, and/or VLDL-C, is an accepted endpoint for treating or preventing cardiovascular disease.

With regard to treatment and prevention of atherosclerosis, it is recognized by practitioners in the field of cardiovascular medicine that atherosclerosis is a progressive disease, marked by the accumulation of atherosclerotic plaque in the lumen of arteries of an individual. Effective treatment of the disease is indicated by retarding (i.e., reducing the rate of) the accumulation of plaque, by arresting the development of plaque, or by reversal of the deposit of plaque. Prevention of atherosclerosis refers to any measure that prevents or prolongs the time before the primary pathological endpoint of atherosclerosis occurs, namely, the complete occlusion of the arterial lumen, which is followed by ischemia and its attendant pathologies. Therefore, data showing reduction of plaque area in controlled experiments (e.g., treated vs. untreated subjects), or regulation of CETP activity, lowering of circulating cholesterol, increase in HDL-C levels, decrease in LDL-C, VLDL-C, or cholesterol levels, increase in HDL-C to LDL-C/VLDL-C/cholesterol ratios are data which are demonstrative of treatment and of prevention of atherosclerosis.

A CETP vaccine composition described herein may be administered to an individual by any route that is compatible for use of the adjuvant(s) included in the vaccine composition. Accordingly, the preferred route of administration is parenterally, including, but not limited to, subcutaneously (s.c.), intramuscularly (i.m.), intravenously (i.v.), intradermally (i.d.), intraperitoneally (i.p.), and intra-arterially (i.a.). A subcutaneous or intravenous route of administration is particularly preferred in some embodiments. Other routes of administration useful according to the methods of the invention include but are not limited to sublingual, intratracheal, inhalation and mucosal routes such as oral, intranasal, ocular, vaginal, and rectal.

The immunostimulatory oligonucleotide and/or antigenic hybrid polypeptide and/or optionally other therapeutic agents may be administered simultaneously or sequentially. When the other therapeutic agents are administered simultaneously they can be administered in the same or separate formulations, but are administered at the same time. The antigenic hybrid polypeptide and optionally other therapeutic agents are administered sequentially with one another and with the immunostimulatory oligonucleotide, when the administration of the antigenic hybrid polypeptide and other therapeutic agents and the immunostimulatory oligonucleotide is temporally separated. The separation in time between the administration of these compounds may be a matter of minutes or it may be longer. Other therapeutic agents include but are not limited to non-nucleic acid adjuvants, cytokines, antibodies, antigens, anti-atherosclerosis agents etc.

A CETP vaccine composition described herein may be formulated for parenteral administration to an individual using a pharmaceutically acceptable vehicle (carrier, buffer) and may further be combined with one or more other pharmaceutically acceptable ingredients that enhance parenteral administration, e.g, by improving the dissolution or suspension of the antigenic hybrid polypeptide and/or adjuvant of the vaccine composition. Preferred pharmaceutically acceptable vehicles may be a phosphate buffered saline or other isotonic, aqueous buffer. By “pharmaceutically acceptable” is meant a material that is not biologically, chemically, or in any other way, incompatible with body chemistry and physiology and also does not adversely affect the properties of the vaccine composition described herein.

Is A CETP vaccine composition described herein may also be combined with or co-administered (i.e., simultaneously or consecutively) with one or more therapeutic agents or vaccines.

Appropriate dosing for use of a vaccine composition described herein can be established using general vaccine methodologies of the art based on measuring parameters for which a particular vaccine composition is proposed to affect (e.g., inhibition of endogenous CETP activity; alteration of level(s) of lipoprotein-associated cholesterol) and the monitoring for potential contraindications. In addition, data available from studies of previously described, peptide-based CETP vaccines (e.g., CETi-1, Avant Immunotherapeutics, Inc., Needham, Mass.) may also be considered in the development of specific dosing parameters for the improved CETP vaccine compositions described herein (see, e.g., Davidson et al., Atherosclerosis, 169: 113-120 (2003)). Human clinical trials involving the administration of CpG oligonucleotides with hepatitis B antigen are described in Cooper, C. L. et al. CpG 7909, an immunostimulatory TLR9 agonist oligodeoxynucleotide, as adjuvant to Engerix-B HBV vaccine in healthy adults: A double-blind Phase I/II study. J Clin. Immunol 24, 693-702 (2004); Halperin, S. A. et al. A phase I study of the safety and immunogenicity of recombinant hepatitis B surface antigen co-administered with an immunostimulatory phosphorothioate oligonucleotide adjuvant. Vaccine 21, 2461-2467 (2003).; Siegrist, C. A. et al. Co-administration of CpG oligonucleotides enhances the late affinity maturation process of human anti-hepatitis B vaccine response. Vaccine 23, 615-622 (2004). Human clinical trials involving the administration of CpG oligonucleotides with anthrax vaccine are described in Rynkiewicz, D. et al. Marked enhancement of antibody response to anthrax vaccine adsorbed with CPG 7909 in healthy volunteers. ICAAC poster presentation. (2005). Human clinical trials involving the administration of CpG oligonucleotides with hepatitis B antigen in HIV positive subjects are described in Cooper, C. L. et al. CPG 7909 adjuvant improves hepatitis B virus vaccine seroprotection in antiretroviral-treated HIV-infected adults. AIDS 19, 1473-1479 (2005). Human clinical trials involving the administration of CpG oligonucleotides with ragweed allergen are described in Creticos, P. S. et al. Immunotherapy with immunostimulatory oligonucleotides linked to purified ragweed Amb a 1 allergen: effects on antibody production, nasal allergen provocation, and ragweed seasonal rhinitis. J Allergy Clin. Immunol. 109(4), 742-743. 2002 and Simons et al., Selective immune redirection in humans with ragweed allergy by injecting Amb a 1 linked to immunostimulatory DNA. J Allergy Clin Immunol 113, 1144-1151 (2004). This clinical trial report demonstrates that the anti-allergic effects of CpG ODN are not limited to mice, but also are seen in humans. Human clinical trials involving the administration of CpG oligonucleotides with cancer antigens are described in van Ojik, H. et al. Phase I/II study with CpG 7909 as adjuvant to vaccination with MAGE-3 protein in patients with MAGE-3 positive tumors. Ann. Oncol. 13(1), 157. 2002. A clinical study involving CpG oligonucleotide and a melan-A antigen was described in Speiser, D. E. et al. Rapid and strong human CD8(+) T cell responses to vaccination with peptide, IFA, and CpG oligodeoxynucleotide 7909. J. Clin. Invest 115, 739-746 (2005).

The vaccine compositions are administered in one or more doses over time, with an initial priming vaccination being followed, typically, by one or more “booster” vaccinations at a later time to raise or maintain an anti-CETP antibody titer. The exact dosing and boosting schedule will be determined by the practitioner to optimize the safety and effectiveness of the vaccine composition for modulating endogenous CETP activity.

As mentioned previously, studies in animal models, especially in rabbits, have been useful in developing previously described CETP vaccine compositions for use in humans, including the monitoring for undesired autoimmune reactions (see, e.g., U.S. Pat. No. 6,555,113; Davidson et al., 2003). Example 1, below, provides studies in rabbits and mice of representative vaccine compositions of the invention in which such vaccine compositions exhibit significant and unexpected enhancement of levels of anti-CETP antibody compared to levels obtained with previously described CETP vaccines and without evidence of significant undesired autoimmune reactions, such as tissue or organ damage, hypersensitivity reaction, injection site reactions (erythema, induration, tenderness), and the like.

A more complete appreciation of this invention and the advantages thereof will be obtained from the following non-limiting examples.

### EXAMPLES

### Example 1

### Effect of Adjuvants on Production of Anti-CETP Antibodies Elicited by Vaccine Compositions in Test Animals

This study compared the effect of CpG oligonucleotide adjuvant, 5′TCGTCGTTTTGTCGTTTTGTCGTT3′, SEQ ID NO.: 3, (Coley Pharmaceutical Group, Inc., Wellesley, Mass.) on the production of anti-CETP antibodies in test animals treated with either of two anti-CETP peptide-based vaccine compositions.

The CETi-1 vaccine (Avant Immunotherapeutics, Inc., Needham, Mass.) employs an acetate salt of a disulfide linked homodimer of a 31-amino acid synthetic peptide (see, U.S. Pat. No. 6,410,022) having the amino acid sequence:

| | |
|---|---|
| CQYIKANSKFIGITEFGFPEHLLVDFLQSLS; | (SEQ ID NO: 1) |

wherein two of the 31 amino acid peptide monomers are linked together via a disulfide bond between the amino-terminal cysteine residues, and wherein the carboxy-terminal serine (S) residue of each monomer has an alpha amide group (—CO—NH₂) as a carboxylic acid blocking group. The sequence of the carboxy-terminal 16 amino acids (in bold) in the above sequence are identical to the carboxy-terminal 16 amino acid residues of human CETP and provide a well known B cell epitope of human CETP (see, e.g., U.S. Pat. No. 6,410,022; U.S. Pat. No. 6,555,113; Davidson et al. (2003)). The underlined 14-amino acid sequence of the amino-terminal portion of the above peptide (i.e., amino acids 2-15 of SEQ ID NO:1) are identical to a sequence of tetanus toxin that has been described as a naturally occurring, broad range or universal helper T cell epitope (“TT 830-843”; see, e.g., Valmori et al., J. Immunol., 149: 717-721 (1992); Alexander et al., Immunity, 1: 751-761 (1994)).

The other CETP vaccine composition employs a polypeptide (designated “CETI-2”) having the amino acid sequence:

| | |
|---|---|
| aKChaVAAWTLKAaFGFPEHLLVDFLQSLS; | (SEQ ID NO: 2) |

wherein “a” is D-alanine and “Cha” is cyclohexylalanine. The CETI-2 polypeptide is a monomer. The sequence of the carboxy-terminal 16 amino acids (in bold) is identical to the carboxy-terminal 16 amino acids of human CETP that define a B cell epitope of human CETP and is the same as that found in the antigenic peptide of the CETi-1 vaccine (see, above). The underlined sequence of the amino-terminal 12 amino acids defines a synthetic “broad range” or “universal” helper T cell epitope referred to as a pan-DR epitope (“PADRE”; IDM Pharma, Inc., Irvine, Calif.).

The study consisted of two parts: (1) an evaluation of intramuscularly administered vaccine compositions in New Zealand White rabbits and (2) an evaluation of subcutaneously administered vaccine compositions in BALB/c mice. The basic study design of each part was similar. Following an initial series of immunizations (administration of vaccine composition), the animals were boosted at week 15-16. Blood samples were taken periodically. Table 1 (rabbits) and Table 2 (mice), below, summarize the regimen for each part of the study.

TABLE 1
Regimen for study of vaccine compositions in Male NZW rabbits

| | Animals | | | Dose | | |
|---|---|---|---|---|---|---|
| | (Serial | Vaccine | | (vaccine | Dosing | |
| Group | Number) | Composition | Route | peptide) | Weeks | Bleed Schedule (Weeks) |
| 1 | 701-708 | CETi-1 + | i.m. | 0.10 mg | 1, 3, 5, 17 | 1, 3, 5, 7, 13, 15, 19, 22, |
| | | alhydrogel | | | | 25, 27, 32, 39, 44 |
| 2 | 709-716 | CETi-1 + | i.m. | 0.10 mg | 1, 3, 5, 17 | 1, 3, 5, 7, 13, 15, 19, 22, |
| | | alhydrogel + | | | | 25, 27, 32, 39, 44 |
| | | CpG adjuvant | | | | |
| 3 | 717-723 | CETI-2 + | i.m. | 0.10 mg | 1, 3, 5, 17 | 1, 3, 5, 7, 13, 15, 19, 22, |
| | | alhydrogel | | | | 25, 27, 32, 39, 44 |
| 4 | 724-730 | CETI-2 + | i.m. | 0.10 mg | 1, 3, 5, 17 | 1, 3, 5, 7, 13, 15, 19, 22, |
| | | alhydrogel + | | | | 25, 27, 32, 39, 44 |
| | | CpG adjuvant | | | | |

i.m. = intramuscular administration

TABLE 2
Regimen for study of vaccine compositions in mice

| | Animals | | | Dose | | |
|---|---|---|---|---|---|---|
| | (Serial | Vaccine | | (vaccine | Dosing | |
| Group | Number) | Composition | Route | peptide) | Week | Bleed Schedule (Week) |
| 1 | 1-10 | CETi-1 + | s.c. | 0.10 mg | 1, 3, 5, 15 | 1, 3, 5, 7, 12, 15, 17, 22, |
| | | alhydrogel | | | | 26, 30, 31, 34, 37, 40 |
| 2 | 11-20 | CETi-1 + | s.c. | 0.10 mg | 1, 3, 5, 15 | 1, 3, 5, 7, 12, 15, 17, 22, |
| | | alhydrogel + | | | | 26, 30, 31, 34, 37, 40 |
| | | CpG adjuvant | | | | |
| 3 | 21-30 | CETI-2 + | s.c. | 0.10 mg | 1, 3, 5, 15 | 1, 3, 5, 7, 12, 15, 17, 22, |
| | | alhydrogel | | | | 26, 30, 31, 34, 37, 40 |
| 4 | 31-40 | CETI-2 + | s.c. | 0.10 mg | 1, 3, 5, 15 | 1, 3, 5, 7, 12, 15, 17, 22, |
| | | alhydrogel + | | | | 26, 30, 31, 34, 37, 40 |
| | | CpG adjuvant | | | | |

s.c. = subcutaneous administration

Rabbits

Thirty (30) Specific Pathogen Free (SPF) New Zealand White male rabbits were obtained from Millbrook Breeding Labs (Amherst, Mass.) weighing approximately 1.5 to 2 kg. The animals were examined for signs of disease or injury upon receipt. The animals were held in quarantine, during which time no abnormal findings were observed.

Each animal was identified with a unique number that was tattooed on the ventral surface of the pinna. Cage labels identified each cage with the study number, sex, species, individual number and study group.

The animals were conventionally housed in individual stainless steel cages. Upon receipt rabbits were placed on a Lab Diet Certified Rabbit Diet (Lab Diet #5322, PMI Nutrition International, Brentwood, Mo.), and fed approximately 125 grams per day. Water was made available ad libitum. Animals were monitored daily for feed and water consumption (qualitatively) and for signs of distress. All husbandry conditions were maintained as described in the Guide for the Care and Use of Laboratory Animals (National Research Council).

Mice

Forty-five (45) Specific Pathogen Free (SPF) Balb/c female mice were obtained from Taconic (Germantown, N.Y.) at 8 to 9 weeks old. The animals were examined for signs of disease or injury upon receipt. The animals were held in quarantine, during which time no abnormal findings were observed.

Each animal was identified with a unique sequence number by ear punches. Cage labels identified each cage with the study number, sex, species, individual numbers and study group.

The animals were conventionally housed in plastic cages. Upon receipt rabbits were placed on a Lab Diet Certified Rodent Diet (Lab Diet #5002, PMI Nutrition International, Brentwood, Mo.), fed ad libitum. Water was made available ad libitum. Animals were monitored daily for feed and water consumption (qualitatively) and for signs of distress. All husbandry conditions were maintained as described in the Guide for the Care and Use of Laboratory Animals (National Research Council).

Test Article Formulations

Materials

The CETi-1 peptide and the CETI-2 peptide were synthesized and obtained commercially (NeoMPS). Each peptide was combined with a 2% alhydrogel suspension (10 mg aluminum/ml) (Superfos; Biosector, Kvistgård, Denmark) in 10×PBS, pH 7.0 (0.5 M sodium phosphate, 1.5M NaCl). CpG oligonucleotide SEQ ID NO: 3 (Coley Pharmaceuticals, Inc.) was obtained from lot ACZ-01F-007-M (21.29 mg/ml) 100 mg; lot ACZ-031-016-M (23.21 mg/ml) 40 mg.

Methods

The formulation of the two vaccine peptides is outlined in the Table 3, below. The two vaccine peptides (CETi-1 peptide and CETI-2 peptide) were reconstituted in 5% acetic acid containing 0.2% Tween-80 to approximately 10 mg/mL. This peptide solution was filtered through a 0.2 μm pore membrane, and the peptide concentration was determined from the absorbance at 275 nm. For rabbits, the vaccine peptides were formulated with alhydrogel in a final proportion of 100 μg of peptide to 750 μg aluminum, +/−1.0 mg of SEQ ID NO: 3 per 500 μL (i.m. dose) as indicated. For mice, the vaccine peptides were formulated with alhydrogel in a final proportion of 100 μg of peptide to 75 μg aluminum, +/−0.10 mg of SEQ ID NO: 3 per 50 μL (s.c. dose) as indicated. All vaccines were prepared no more than 24 hours before being administered to animals.

TABLE 3
Peptide vaccine formulations

| Ingredient | Volume | |
|---|---|---|
| 10X PBS | 0.100 | mL |
| Stock peptide in 5% acetic acid | 0.100 | mL* |
| 10 N NaOH | 8 | μL |
| alhydrogel (10.6 mg Al + 3/mL) | 0.142 | mL |
| WFI | Q.S. to 1 | mL |

*For example, 39 μL peptide at 12.8 mg/mL + 61 μL 5% acetic acid
WFI = water for injection

All procedures involving animals were approved by the Institutional Animal Care and Use Committee at AVANT Immunotherapeutics. All procedures took place in the facilities of AVANT Immunotherapeutics (Needham, Mass.).

The rabbits were weighed before each blood sample was taken. Blood samples were taken on the days indicated in Table 1 from the marginal ear vein. Blood was processed as serum. Serum samples were stored at −70° C. until use. Prior to dosing, the skin covering the injection site was shaved using an electric clipper. The vaccine preparations was gently mixed, and then drawn up into a needle and syringe. The animal was gently restrained, and the vehicle (no peptide) or vaccine composition was injected intramuscularly (i.m.) into alternating thigh muscles according to the details presented in Table 1.

Blood samples from mice were taken on the days indicated in Table 2 from the retroorbital sinus. Blood was processed as serum. Serum samples were stored at −70° C. until use. For mice, prior to dosing, the skin covering injection site was shaved using an electric clipper. The vaccine preparations was gently mixed, and then drawn up into a needle and syringe. The animal was gently restrained, and the vehicle or vaccine was injected subcutaneously (s.c.) at the base of the tail according to the details presented in Table 2.

Assay for detection of rabbit antibodies specific for human CETP

An ELISA for antibodies that bind human CETP utilizes a direct coat of recombinant human CETP whole protein to detect antibodies in rabbit serum or plasma.

ELISA Reagents

Recombinant human CETP coating (whole protein).

Horseradish peroxidase-conjugated, affinity purified, goat anti-rabbit IgG (H+L).

10× Dulbecco's Phosphate-Buffered Saline (PBS), calcium chloride-free, magnesium chloride-free (Gibco Cat. # 1420075, 500 ml bottle).

CETi Assay Buffer, see below.

Control Curve: Purified rabbit anti-CETP serum, starting concentration 1 μg/mL serially diluted 1:2. Pre-made and frozen at −70° C.

TMB Peroxidase Substrate System (Kirkegaard & Perry, 50-76-00).

2N H₂SO₄ Stop Solution, see below.

Wash Buffer (1×DPBS/0.05% Tween 20).

0.124 M carbonate coating buffer, pH 10.0, see below.

Proclin 300 (Supleco Cat # 4-8127), or equivalent.

Igepal CA-630 (Sigma Cat # 1-3021), or equivalent.

Triton X-100 (J. T. Baker cat #198-07), or equivalent

Non-fat dry milk (Biorad Cat # 170-6404), or equivalent.

Tween 20 (J. T Baker Cat #X251-07), or equivalent.

Sulfuric Acid (J. T Baker Cat #9681-04), or equivalent.

BSA (bovine serum albumin).

Preparation of Solutions and Reagents

For preparing 0.124 M carbonate, pH10.0: 100 mL of a 0.5 M sodium carbonate anhydrous (FW=105.99) solution (5.2 g/100 mL) and 100 mL of a 0.5 M sodium bicarbonate (FW=84.01) solution (4.2 g/100 mL) were prepared in distilled water and filter sterilized through a 0.22 μm (“micron”) pore membrane. The solutions were labeled and stored at room temperature (RT).

To make 50 mL of 0.124 M Carbonate (pH 10.0), 6.8 ml of the carbonate solution was mixed with 5.6 mL of the bicarbonate solution and 37.6 mL distilled water, filtered sterilized through a 0.22 micron membrane, then labeled and stored at RT.

Recipe for CETi Assay Buffer

For a total volume of 1 liter of CETi Assay Buffer, the following were added to 1×PBS: 5 mL of the aqueous cold water fish gelatin, 6 mL of Igepal CA-630, 9 mL of Triton X-100, 10 mL of Proclin 300, and 10 g of BSA. This was mixed on a stir plate with low heat until in solution, then 10 g non-fat dry milk was added and mixed thoroughly until there were no clumps of milk left (the mixture remained opaque but looked homogeneous). QS to 1 liter with 1×PBS, poured into a clean 1 liter bottle with a screwcap and labeled appropriately. This buffer was stored at 4° C.

Recipe of Wash Buffer, 10× and 1×:

For 10× Wash Buffer. For each 500 mL of 10× Wash Buffer to be made, 2.5 mL of Tween-20 was added to the 500 mL Gibco 10×DPBS bottle and mixed by shaking.

For 1× wash buffer: 10× Wash Buffer stock was diluted to 1× with distilled water.

Recipe for 2N₂HS0₄ Stop Solution:

For a total volume of 1 liter of Stop Solution, 5.5 mL of Sulfuric Acid was added to 944.45 mL of water. The solution was mixed on a stir plate until the solution cooled down to approximately room temperature. This solution was stored at room temperature.

ELISA Protocol

1. Coat 96-well microtiter plates the day before the assay is to be performed with 5 μg/mL human CETP whole protein in carbonate coating buffer. Add to plate at 100 μl/well. Seal and label plate with human CETP, date and initials. Store at 4° C., overnight.

2. Empty the plate (contents) into the sink and bang dry.

3. Block the human CETP pre-coated plate with 250 μL CETP Assay Buffer, seal and shake at 150 rpm for 2-9 hours at room temperature.

4. Remove one set of the Control Curve samples from the freezer 30-60 minutes prior to adding them to the plate. Allow them to thaw and warm to room temperature; vortex before use.

5. Dilute all samples in CETi Assay Buffer. Start with a 1:100 dilution for an initial screen. For titering purposes dilute either in bullets or in the plate.

6. Empty blocking buffer and wash plate 3× and bang dry.

7. Transfer 100 μL/well of each control and or sample in duplicate into the blocked plate. Seal the plate, and incubate for 1.5 hours at room temperature while shaking at 150 rpm. At the end of the incubation, aspirate/wash the plate 4× and bang dry.

8. When it is less than 15 minutes from the end of the 1.5 hour sample incubation, remove an aliquot of the pre-titered goat-anti-rabbit-HRP conjugate from the −70° C. freezer and thaw. Make a 1:100 intermediate dilution in CETi Assay Buffer. From the 1:100 dilution, dilute to the working concentration or titer, which is recorded on the stock label (1:50,000). Add 100 μL/well of the conjugate, seal the plate, and incubate for 1 hour at room temperature, while shaking at 150 rpm.

At a point 15 minutes or less from the end conjugate incubation, mix the TMB Peroxidase Substrate and Solution B, at a 1:1 ratio. Mix thoroughly.

9. At the end of the hour conjugate incubation, aspirate/wash the plate 4× in wash buffer, bang dry. Add 100 μL of the TMB mix to the wells. Incubate for 30 minutes in the dark (e.g., in a drawer, or under aluminum foil) at room temperature with no shaking.

10. Stop the reaction at exactly 30 minutes with 50 μL/well of 2N H₂SO₄. Read as soon as possible at 450 nm (no longer than 15 minutes).

11. Titer was determined as the inverse of the greatest dilution that yielded an absorbance at 450 nm that was 3 times over that of a pre-vaccination sample from the same animal. A sample was considered positive if it yields an absorbance 3 times the pre-vaccination sample at a dilution of 1: 100.

Assay for Detection of Mouse Antibodies Specific for Human CETP

This ELISA utilized a direct coat of recombinant human CETP whole protein to detect antibodies in mouse serum or plasma.

The procedure for the mouse sample ELISA was the same as described above for the rabbit samples, with the following changes in reagents:

1. Horseradish peroxidase-conjugated, affinity purified, goat anti-rabbit IgG (H+L) was replaced with horseradish peroxidase-conjugated, affinity purified, goat anti-mouse IgG.

2. Control Curve: purified rabbit anti-CETP serum, starting concentration 1 μg/mL serially diluted 1:2, pre-made and frozen at −70° C. was replaced with purified mouse monoclonal anti-CETP antibody TP2, starting concentration of 1 μg/mL, serially diluted 1:2, pre-made and frozen at −70° C.

Injection Site Reactogenicity

Rabbits and mice were examined for reactogenicity at the site of injection for 7 days following each injection. The following scale was used to determine and report the level of reactogenicity in each animal.

TABLE 4
Reactogenicity scale

| Score | Grade | Erythema | Swelling |
|---|---|---|---|
| 0 | none | normal color | No swelling |
| 1 | minimal | light pink; indistinct | Slight swelling; indistinct |
| | | | border |
| 2 | mild | bright pink or pale red; | Defined swelling; distinct |
| | | distinct | border |
| 3 | moderate | bright red | Defined swelling; raised |
| | | | border (approx. 1 mm) |
| 4 | severe | dark red; pronounced | Pronounced swelling; raised |
| | | | border (approx. >1 mm) |

Statistics

Limited statistical analysis of the geometric mean data was performed, as appropriate.

Results

The antibody titers for individual animals and group mean titers (GMT) are provided in Tables 5 and 6, below. A summary of the statistical analysis of the data follow in Tables 7 and 8, below.

TABLE 5
Rabbit anti-CETP antibody titer data

| | Animal | Antibody Titer | Antibody Titer |
|---|---|---|---|
| Group* | (Serial Number) | (Post Prime) | (Post Boost) |
| 1 | 701 | 1000 | 8000 |
| 1 | 702 | 16000 | 16000 |
| 1 | 703 | 16000 | ** |
| 1 | 704 | 4000 | 8000 |
| 1 | 705 | 2000 | 8000 |
| 1 | 706 | 1000 | 8000 |
| 1 | 707 | 32000 | 128000 |
| 1 | 708 | 8000 | 16000 |
| 1 | GMT*** | 5187.36 | 14491.58 |
| 2 | 709 | 32000 | 32000 |
| 2 | 710 | 8000 | 64000 |
| 2 | 711 | 16000 | 128000 |
| 2 | 712 | 2000 | 32000 |
| 2 | 713 | 4000 | 32000 |
| 2 | 714 | 8000 | 128000 |
| 2 | 715 | 16000 | 256000 |
| 2 | 716 | 32000 | 128000 |
| 2 | GMT | 10374.72 | 76109.26 |
| 3 | 717 | 32000 | 64000 |
| 3 | 718 | 32000 | 128000 |
| 3 | 719 | 32000 | 128000 |
| 3 | 720 | 64000 | 128000 |
| 3 | 721 | 64000 | 64000 |
| 3 | 722 | 32000 | 16000 |
| 3 | 723 | 128000 | 256000 |
| 3 | GMT | 47551.82 | 86137.61 |
| 4 | 724 | 32000 | 512000 |
| 4 | 725 | 128000 | 1024000 |
| 4 | 726 | 64000 | 512000 |
| 4 | 727 | 32000 | 256000 |
| 4 | 728 | 128000 | 512000 |
| 4 | 729 | 64000 | 512000 |
| 4 | 730 | 32000 | 256000 |
| 4 | GMT | 57966.31 | 463730.56 |

*Group 1 = CETi-1 + alhydrogel; Group 2 = CETi-1 + alhydrogel + SEQ ID NO: 3; Group 3 = CETI-2 + alhydrogel; Group 4 = CETI-2 + alhydrogel + SEQ ID NO: 3.
**Rabbit 703 was euthanized after sustaining an injury.
***GMT = group mean titer.

TABLE 6
Mouse anti-CETP antibody titer data

| | Animal | Antibody Titer | Antibody Titer |
|---|---|---|---|
| Group | (Serial Number) | (Post Prime) | (Post Boost) |
| 1 | 1 | 50 | 8000 |
| 1 | 2 | 50 | 1000 |
| 1 | 3 | 50 | 500 |
| 1 | 4 | 400 | 250 |
| 1 | 5 | 400 | 4000 |
| 1 | 6 | 50 | 2000 |
| 1 | 7 | 400 | 500 |
| 1 | 8 | 50 | 125 |
| 1 | 9 | 50 | 1000 |
| 1 | 10 | 3200 | 3200 |
| 1 | GMT | 141.42 | 1048.12 |
| 2 | 11 | 800 | 8000 |
| 2 | 12 | 800 | 32000 |
| 2 | 13 | 800 | 4000 |
| 2 | 14 | 100 | 32000 |
| 2 | 15 | 50 | 1000 |
| 2 | 16 | 2000 | 16000 |
| 2 | 17 | 200 | 16000 |
| 2 | 18 | 1000 | 4000 |
| 2 | 19 | 2000 | 32000 |
| 2 | 20 | 400 | 8000 |
| 2 | GMT | 491.29 | 9849.16 |
| 3 | 21 | 200 | 4000 |
| 3 | 22 | 200 | 4000 |
| 3 | 23 | 100 | 16000 |
| 3 | 24 | 50 | 2000 |
| 3 | 25 | 200 | 8000 |
| 3 | 26 | 100 | 2000 |
| 3 | 27 | 800 | 4000 |
| 3 | 28 | 50 | 2000 |
| 3 | 29 | 50 | 125 |
| 3 | 30 | 50 | 125 |
| 3 | GMT | 114.87 | 2000 |
| 4 | 31 | 2000 | 16000 |
| 4 | 32 | 8000 | 256000 |
| 4 | 33 | 1600 | 128000 |
| 4 | 34 | 8000 | 32000 |
| 4 | 35 | 64000 | 16000 |
| 4 | 36 | 64000 | 8000 |
| 4 | 37 | 1600 | 32000 |
| 4 | 38 | 1600 | 2000 |
| 4 | 39 | 4000 | 16000 |
| 4 | 40 | 1600 | 32000 |
| 4 | GMT | 5173.82 | 24251.47 |

Group 1 = CETi-1 + alhydrogel; Group 2 = CETi-1 + alhydrogel + SEQ ID NO: 3; Group 3 = CETI-2 + alhydrogel; Group 4 = CETI-2 + alhydrogel + SEQ ID NO: 3.
GMT = group mean liter.

TABLE 7
Statistical analysis of rabbit data

| | Group | Geometric | | Statistically |
|---|---|---|---|---|
| Group | Number | Mean | 95% CI | Different From |
| CETi-1 + | 1 | 14491.58 | (7631.20, | Groups 2, 3, 4 |
| alhydrogel | | | 27446.67) | |
| CETi-1 + | 2 | 76109.26 | (41772.77, | Groups 1, 4 |
| alhydrogel + | | | 138690.48) | |
| CpG adjuvant | | | | |
| CETI-2 + | 3 | 86137.61 | (45706.69, | Groups 1, 4 |
| alhydrogel | | | 162754.79) | |
| CETI-2 + | 4 | 463730.52 | (245241.81, | Groups 1, 2, 3 |
| alhydrogel + | | | 873269.94) | |
| CpG adjuvant | | | | |

TABLE 8
Statistical analysis of mouse data

| | Group | Geometric | | Statistically |
|---|---|---|---|---|
| Group | Number | Mean | 95% CI | Different From |
| CETi-1 + | 1 | 1048.12 | (437.03, | Groups 2, 4 |
| alhydrogel | | | 2514.93) | |
| CETi-1 + | 2 | 9849.16 | (4105.16, | Groups 1, 3 |
| alhydrogel + | | | 23623.56) | |
| CpG adjuvant | | | | |
| CETI-2 + | 3 | 2000.00 | (837.15, | Groups 2, 4 |
| alhydrogel | | | 4769.52) | |
| CETI-2 + | 4 | 24251.47 | (10198.54, | Groups 1, 3 |
| alhydrogel + | | | 58104.59) | |
| CpG adjuvant | | | | |

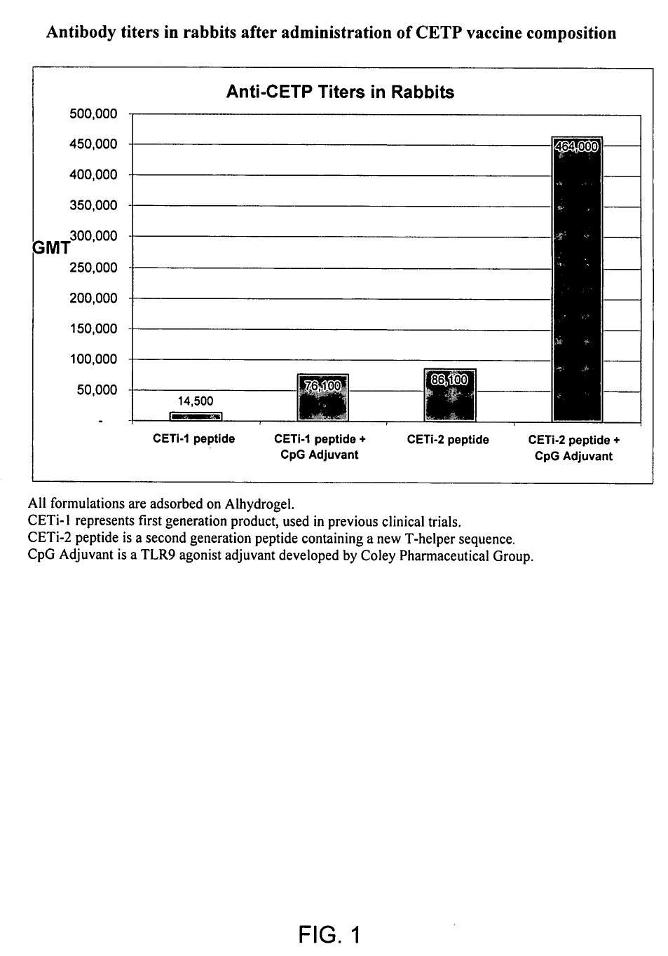

A bar graph prepared from the data of Table 7 is presented as FIG. 1.

Reactogenic results No evidence of any reactogenicity at sites of administration was observed during the 7 days following administration of vaccine composition.

Anti-CETP antibody responses As seen by the foregoing results, co-administration of CpG adjuvant with alhydrogel-adsorbed CETP vaccine peptide (CETi-1 or CETI-2) elicited increased titers of anti-human CETP antibodies compared to the administration of alhydrogel-adsorbed CETP vaccine peptide in the absence of CpG adjuvant in both NZW rabbits and BALB/c mice.

In rabbits, co-administration of CpG adjuvant with either CETP vaccine peptide elicited higher antibody titers both post-prime (a 2-fold or less increase) and post-boost (a 5-fold increase) compared to administration of alhydrogel-adsorbed CETi-1 vaccine peptide or alhydrogel CETI-2 vaccine peptide in the absence of CpG adjuvant.

In mice, co-administration of CpG adjuvant with either CETP vaccine peptide elicited higher antibody titers both post-prime (a 3 to 45-fold increase) and post-boost (a greater than 7-fold increase) compared to administration of alhydrogel-adsorbed CETi-1 peptide or alhydrogel CETI-2 vaccine peptide without CpG adjuvant.

Compared to the clinical vaccine formulation in which CETi-1 vaccine peptide is adsorbed to alhydrogel, the CETI-2 vaccine peptide adsorbed to alhydrogel and co-administered with CpG adjuvant elicited a greater than 20 times higher anti-CETP antibody titer in mice and a greater than 30 times higher anti-CETP antibody titer in rabbits+post-boost.

The data indicate that co-administration of the CpG adjuvant significantly enhanced the anti-CETP antibody response elicited by the current clinical CETi-1 vaccine composition. Co-administration of the CpG adjuvant also significantly enhanced the anti-CETP antibody response elicited by the CETI-2 vaccine peptide adsorbed to alhydrogel.

All patents, articles, and publications cited herein are incorporated herein by reference.

Although a number of embodiments have been described above, it will be understood by those skilled in the art that modifications and variations of the described compositions and methods may be made without departing from either the spirit of the invention or the scope of appended claims.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A vaccine composition for eliciting antibodies in an individual to endogenous cholesteryl ester transfer protein (CETP) comprising:
(a) an antigenic hybrid polypeptide comprising a B cell epitope portion linked to a helper T cell epitope portion, wherein said B cell epitope portion comprises a B cell epitope of said endogenous CETP, and wherein said helper T cell epitope portion comprises a broad range helper T cell epitope that binds multiple class II major histocompatibility complex (MHC) alleles expressed on antigen-presenting cells, and, optionally, wherein said broad range helper T cell epitope has a non-naturally occurring amino acid sequence and binds multiple DR alleles expressed on antigen-presenting cells, and
(b) an adjuvant comprising an immunostimulatory oligonucleotide.

2. The vaccine composition according to claim 1, wherein the immunostimulatory oligonucleotide is a DNA CpG oligonucleotide having at least one unmethylated CpG dinucleotide.

3. The vaccine composition according to claim 2, wherein said broad range helper T cell epitope is a peptide selected from the group consisting of broad range helper T cell epitope peptides derived from tetanus toxoid, diphtheria toxoid, pertussis vaccine, Bacile Calmette-Guerin (BCG), polio vaccine, measles vaccine, mumps vaccine, rubella vaccine, purified protein derivative of tuberculin, keyhole limpet hemocyanin, hsp 65, hsp70, and combinations thereof.

4. The vaccine composition according to claim 2, wherein said broad range helper T cell epitope is a non-naturally occurring peptide that binds multiple DR alleles expressed on antigen-presenting cells.

5. The vaccine composition according to claim 4, wherein said broad range helper T cell epitope comprises the amino acid sequence:

| | |
|---|---|
| aKChaVAAWTLKAa, | (amino acids 1-12 of SEQ ID NO: 2) |

wherein “a” is D-alanine and “Cha” is cyclohexylalanine.

6. The vaccine composition according to claim 2, wherein said broad range helper T cell epitope has an amino acid sequence selected from the group consisting of:

| | |
|---|---|
| QYIKANSKFIGITE | (amino acids 2-15 of SEQ ID NO: 1) |
| and | |
| aKChaVAAWTLKAa, | (amino acids 1-12 of SEQ ID NO: 2) |

wherein “a” is D-alanine and “Cha” is cyclohexylalanine.

7. The vaccine composition according to claim 2, wherein said B cell epitope of CETP has the amino acid sequence:

| | |
|---|---|
| FGFPEHLLVDFLQSLS. | (amino acids 16-31 of |
| | SEQ ID NO: 1) |

8. The vaccine composition according to claim 2, wherein said antigenic hybrid polypeptide is a polypeptide having an amino acid sequence selected from the group consisting of:

| | |
|---|---|
| CQYIKANSKFIGITEFGFPEHLLVDFLQSLS | (SEQ ID NO: 1) |
| and | |
| aKChaVAAWTLKAaFGFPEHLLVDFLQSLS, | (SEQ ID NO: 2) |

wherein “a” is D-alanine and “Cha” is cyclohexylalanine.

9. The vaccine composition according to claim 2, comprising two or more antigenic hybrid polypeptides.

10. The vaccine composition according to claim 8, comprising a dimer of an antigenic hybrid polypeptide that has the following amino acid sequence:

| | |
|---|---|
| CQYIKANSKFIGITEFGFPEHLLVDFLQSLS. | (SEQ ID NO: 1) |

11. The vaccine composition according to claim 2, comprising one or more broad range helper T cell epitopes and one or more B cell epitopes of CETP.

12. The vaccine composition according to claim 2, wherein said helper T cell epitope portion is linked directly to said B cell epitope portion via a peptide bond.

13. The vaccine composition according to claim 2, wherein said helper T cell epitope portion is linked to said B cell epitope portion via a linker peptide.

14. The vaccine composition according to claim 13, wherein said linker peptide consists of 20 or fewer amino acids, wherein more than half of said amino acids are glycine.

15. The vaccine composition according to claim 2, wherein one or more antigenic hybrid polypeptides are linked to a common carrier.

16. The vaccine composition according to claim 2, wherein one or more helper T cell epitope portions and one or more B cell epitope portions are linked to a common carrier.

17. The vaccine composition according to claim 16, wherein said composition further includes a second adjuvant which is aluminum hydroxide.

18. The vaccine composition according to claim 16, wherein said aluminum hydroxide is in the form of a colloidal suspension.

19. The vaccine composition according to claim 2, wherein said CpG oligonucleotide is conjugated to said antigenic hybrid polypeptide.

20. The vaccine composition according to claim 2, wherein said CpG oligonucleotide is an A class oligonucleotide.

21. The vaccine composition according to claim 2, wherein said CpG oligonucleotide is a B class oligonucleotide.

22. The vaccine composition according to claim 21, wherein said B class oligonucleotide has the sequence 5′ TCN₁TX₁X₂CGX₃X₄ 3′ wherein X₁ is G or A X₂ is T, G, or A, X₃ is T or C and X₄ is T or C and N is any nucleotide and N₁ and N₂ are nucleic acid sequences composed of from about 0-25 N's each.

23. The vaccine composition according to claim 2, wherein said CpG oligonucleotide is a C class oligonucleotide.

24. The vaccine composition according to claim 2, wherein said CpG oligonucleotide is a P class oligonucleotide.

25. The vaccine composition according to claim 2, wherein said CpG oligonucleotide is a T class oligonucleotide.

26. The vaccine composition according to claim 2, wherein said CpG oligonucleotide comprises at least one 3′-3′ linkage.

27. The vaccine composition according to claim 2, wherein said CpG oligonucleotide comprises at least one 5′-5′ linkage.

28. The vaccine composition according to claim 2, further comprising a non-nucleotidic brancher moiety.

29. The vaccine composition according to claim 2, further comprising a nucleotidic brancher moiety.

30. The vaccine composition according to claim 2, further comprising a brancher moiety, wherein said CpG oligonucleotides has at least two 5′-ends.

31. The vaccine composition according to claim 2, wherein at least one nucleotide of said CpG oligonucleotide has a stabilized linkage.

32. The vaccine composition according to claim 31, wherein the stabilized linkage is phosphorothioate, phosphorodithioate, methylphosphonate, methylphosphonothioate, boranophosphonate, phosphoramidate, or a dephospho linkage.

33. The vaccine composition according to claim 2, wherein said CG dinucleotide has a phosphodiester or phosphodiester-like internucleotide linkage, and wherein the oligonucleotide includes at least one stabilized internucleotide linkage.

34. The vaccine composition according to claim 2, wherein said CG dinucleotide has a phosphorothioate linkage.

35. The vaccine composition according to claim 2, wherein said CpG oligonucleotide has at least three CG dinucleotides.

36. The vaccine composition according to claim 35, wherein each of said at least three CG dinucleotides has a phosphodiester or phosphodiester-like internucleotide linkage, and wherein the oligonucleotide includes at least one stabilized internucleotide linkage.

37. The vaccine composition according to claim 36, wherein all other nucleotides have a phosphorothioate linkage.

38. The vaccine composition according to claim 2, wherein all nucleotides of said CpG oligonucleotide have a phosphorothioate linkage.

39. The vaccine composition according to claim 2, wherein said CpG oligonucleotide is 5′TCGTCGTTTTGTCGTTTTGTCGTT3′ (SEQ ID NO.: 3).

40. The vaccine composition according to claim 39, wherein all nucleotides of said CpG oligonucleotide have a phosphorothioate linkage.

41. The vaccine composition according to claim 1, wherein the immunostimulatory oligonucleotide is an RNA oligonucleotide.

42. The vaccine composition according to claim 41, wherein the RNA oligonucleotide is 5′-C/U-U-G/U-U-3′,5′-R-U-R-G-Y-3′,5′-G-U-U-G-B-3′, 5′-G-U-G-U-G/U-3′, or 5′-G/C-U-A/C-G-G-C-A-C-3′, wherein C/U is cytosine (C) or uracil (U), G/U is guanine (G) or U, R is purine, Y is pyrimidine, B is U, G, or C, G/C is G or C, and A/C is adenine (A) or C.

43. The vaccine composition according to claim 42, wherein 5′-C/U-U-G/U-U-3′ is CUGU, CUUU, UUGU, or UUUU.

44. The vaccine composition according to claim 42, wherein 5′-R-U-R-G-Y-3′ is GUAGU, GUAGC, GUGGU, GUGGC, AUAGU, AUAGC, AUGGU, or AUGGC.

45. The vaccine composition according to claim 41, wherein the RNA oligonucleotide is GUAGUGU.

46. The vaccine composition according to claim 41, wherein the RNA oligonucleotide is GUGUUUAC.

47. The vaccine composition according to claim 42, wherein 5′-G/C-U-A/C-G-G-C-A-C-3′ is GUAGGCAC, GUCGGCAC, CUAGGCAC, or CUCGGCAC.

48. A method of treating atherosclerosis in an individual comprising administering to said individual
(a) an antigenic hybrid polypeptide comprising a B cell epitope portion linked to a helper T cell epitope portion, wherein said B cell epitope portion comprises a B cell epitope of said endogenous CETP, and wherein said helper T cell epitope portion comprises a broad range helper T cell epitope that binds multiple class II major histocompatibility complex (MHC) alleles expressed on antigen-presenting cells, and, optionally, wherein said broad range helper T cell epitope has a non-naturally occurring amino acid sequence and binds multiple DR alleles expressed on antigen-presenting cells, and
(b) an adjuvant comprising an immunostimulatory oligonucleotide in an effective amount to treat atherosclerosis.

49. A method of increasing the level of high density lipoprotein-associated cholesterol (HDL-C) in the blood of an individual comprising administering to said individual
(a) an antigenic hybrid polypeptide comprising a B cell epitope portion linked to a helper T cell epitope portion, wherein said B cell epitope portion comprises a B cell epitope of said endogenous CETP, and wherein said helper T cell epitope portion comprises a broad range helper T cell epitope that binds multiple class II major histocompatibility complex (MHC) alleles expressed on antigen-presenting cells, and, optionally, wherein said broad range helper T cell epitope has a non-naturally occurring amino acid sequence and binds multiple DR alleles expressed on antigen-presenting cells, and
(b) an adjuvant comprising an immunostimulatory oligonucleotide in an effective amount to increase the level of HDL-C in the blood.

50. A method of increasing the ratio of high density lipoprotein-associated cholesterol (HDL-C) to low density lipoprotein-associated cholesterol (LDL-C), very low density lipoprotein-associated cholesterol (VLDL-C), or total cholesterol in the blood of an individual comprising administering to said individual
(a) an antigenic hybrid polypeptide comprising a B cell epitope portion linked to a helper T cell epitope portion, wherein said B cell epitope portion comprises a B cell epitope of said endogenous CETP, and wherein said helper T cell epitope portion comprises a broad range helper T cell epitope that binds multiple class II major histocompatibility complex (MHC) alleles expressed on antigen-presenting cells, and, optionally, wherein said broad range helper T cell epitope has a non-naturally occurring amino acid sequence and binds multiple DR alleles expressed on antigen-presenting cells, and
(b) an adjuvant comprising an immunostimulatory oligonucleotide
in an effective amount to increase the ratio of HDL-C to LDL-C, VLDL-C, or total cholesterol in the blood.

51. A method of inhibiting CETP activity in an individual comprising administering to said individual
(a) an antigenic hybrid polypeptide comprising a B cell epitope portion linked to a helper T cell epitope portion, wherein said B cell epitope portion comprises a B cell epitope of said endogenous CETP, and wherein said helper T cell epitope portion comprises a broad range helper T cell epitope that binds multiple class II major histocompatibility complex (MHC) alleles expressed on antigen-presenting cells, and, optionally, wherein said broad range helper T cell epitope has a non-naturally occurring amino acid sequence and binds multiple DR alleles expressed on antigen-presenting cells, and
(b) an adjuvant comprising an immunostimulatory oligonucleotide
in an effective amount to inhibit CETP activity.

52. The method of claim 48, wherein the individual is administered a vaccine composition according to claim 1.

53. A method of inhibiting CETP activity in an individual comprising administering to said individual a vaccine composition according to claim 1.

54. A method of inhibiting CETP activity in an individual comprising administering to said individual a vaccine composition according to claim 2.

55. The method according to claim 53, wherein the immunostimulatory oligonucleotide and antigenic hybrid polypeptide are administered simultaneously.

56. The method according to claim 53, wherein the immunostimulatory oligonucleotide and antigenic hybrid polypeptide are administered sequentially.
